(19) United States
(12) Patent Application Publication
(10) Pub. No.: US 2014/0294856 A1
(43) Pub. Date: Oct. 2, 2014

(54) COMBINATION TREATMENT COMPRISING A HDAC6 INHIBITOR AND AN AKT INHIBITOR

(71) Applicants: Eric O. Aboagye, London (GB); Maciej A. Kaliszczak, London (GB)

(72) Inventors: Eric O. Aboagye, London (GB); Maciej A. Kaliszczak, London (GB)

(21) Appl. No.: 14/128,144

(22) PCT Filed: Jun. 21, 2012
(86) PCT No.: PCT/GB2012/051443

§ 371 Date: Jun. 12, 2014

(30) Foreign Application Priority Data
Jun. 22, 2011 (GB) 1110546.7

(51) Int. Cl. A61K 31/437; A61K 45/06; A61K 31/7064; A61K 31/185

(52) U.S. Cl. CPC A61K 31/437; A61K 31/185; A61K 45/06; A61K 31/7064
USPC 424/158.1; 514/293; 514/21.1; 514/44 A; 514/44 R

(57) ABSTRACT

The invention provides compositions comprising a Histone Deacetylase 6 (HDAC6) inhibitor and an AKT inhibitor, pharmaceutical compositions/formulations and kits of parts comprising the same; optionally further comprising one or more anti-cancer agents. The compositions of the invention provide for surprisingly efficacious killing of inter alia, cancer cells. Thus, the invention also provides for the use of the compositions, pharmaceutical compositions and kits of parts in medicine, and further, for use in the prevention or treatment of cancer and/or neuro-degenerative conditions, including Alzheimer's disease, Parkinson's disease, Huntington's disease, Amyotrophic lateral sclerosis (ALS), spinal and bulbar muscular atrophy (SbMA), Rubinstein-Taybi syndrome, Rett syndrome, and Friedreich's ataxia, or autoimmune diseases, including Rheumatoid Arthritis, Myasthenia Gravis, and Multiple Sclerosis.

The invention relates to new compositions, pharmaceutical compositions and medical uses thereof, particularly for use in the treatment of cancers and neurodegenerative disorders.

Inhibitors of certain members of the phosphatidyl inositol 3-OH kinase (PI3K) signalling pathway and general inhibitors of histone deacetylases (HDACs) have previously been combined with the purpose of enhancing cell death and potentially providing anti-cancer therapies (e.g. Rahmani et al (2003) Oncogene 22: 6231-42).

Zinc-dependent histone deacetylases (HDACs) catalyse the removal of acetyl groups from histone tails and also from many non-histone proteins, for example the transcription factor FOXP3 (Wang et al (2009) Nat. Rev. Drug Discov. 8(12): 969-81). A number of HDAC inhibitors have been trialled as potential cancer therapies (see, for example, Kelly & Marks (2005) Nature Clinical Practice Oncology. 2, 150-157; Bolden et al, (2006) Nature Reviews Drug Discovery 5, 769-784; Tan et al. (2010). Journal of Hematology & Oncology, 3:5; and Wagner et al, (2010) Clin Epigenetics. 1(3-4): 117-136) and it is also now apparent that certain HDAC inhibitors have important anti-inflammatory or immunosuppressive effects that may be of therapeutic benefit in immuno-inflammatory disorders or post-transplantation (Wang et al, supra).

There are 18 HDACs which are classified structurally into class I (HDAC1, HDAC2, HDAC3, HDAC8), class IIa (HDAC4, HDAC5, HDAC7, HDAC9), class IIb (HDAC6, HDAC10), class III (sirtuins; SIRT1-7) and class IV (HDAC11) groups (Yang & Seto (2008) Nature Rev. Mol. Cell Biol. 9: 206-218; Haberland et al. (2009) Nature Rev. Genet. 10: 32-42). Class III HDACs or sirtuins act by a nicotinamide-dependent mechanism and are structurally and functionally distinct from class I, II and IV HDAC metalloenzymes. Little is known about HDAC11, the sole class IV member, other than it inhibits expression of interleukin (IL)-10 by dendritic cells in vitro (Villagra et al. (2009) Nature Immunol. 10: 92-100).

Class I HDACs are expressed in all cells and are essential for cell differentiation by contributing to a closed chromatin state and suppression of gene transcription; for example, a cell can become a lymphocyte or a myocyte by turning off genes that promote neuronal or endothelial differentiation, and class I HDACs have a major role in this suppression.

Class II HDACs have more limited cellular expression and often control regulatory processes in a gradual or more subtle manner than their class I counterparts. Both class IIb HDACs (HDAC6 and HDAC10) are distinct from the other HDAC classes in that they have two catalytic domains and can be detected within the nucleus and the cytoplasm (Fontenot et al. (2003) Nature Immunol. 4: 330-336; Wang et al. (2009) Cell 138: 1019-1031). HDAC6 is a unique HDAC in that it has a cytoplasmic location, a ubiquitin-binding site, and it selectively deacetylases alpha-tubulin, Hsp-90 and peroxiredoxin (Prx) I and II (Parmigiani et al. (2008) Proc. Nat. Acad. Sci. USA 105(28): 9633-8).

Dompierre et al (2007) J. Neuroscience 27(13): 3571-83 demonstrated that inhibition of HDAC6 showed a neuroprotective effect in Huntington's Disease (HD). Inhibition of HDAC6 by Trichostatin A (TSA) increased acetylation of alpha-tubulin at Lysine 40 thereby increasing vesicular transport of brain-derived neurotrophic factor (BDNF) compensating for reduced tubulin acetylation in HD brain. Kazantsev & Thompson (2008) Nat. Rev. Drug Disc. 7: 854-68 reviews the state of development of HDAC-based therapeutics and their application for the treatment of human brain disorders.

Rahmani et al (2003) Oncogene 22: 6231-42 reported that the inhibition of PI-3 kinase (PI3K) sensitised human leukemic cells to HDAC inhibitor-mediated apoptosis through p44/42 MAP kinase inactivation and abrogation of p21^{CIP1/WAF1} induction rather than AKT inhibition. The fate of individual cells in response to environmental damage is tightly controlled by the balance between survival and death pathways. Phosphatidyl inositol 3-OH kinase (PI3K) is one of the major signalling molecules that protect cells from apoptosis in response to a variety of noxious stimuli, including chemotherapeutic drugs. Activated PI3K phosphorylates the 3′-OH position of phosphatidyl inositol (PI), PI(4)P, and PI(4,5)P2, generating three signalling phospholipids: PI(3)P, PI(3-4)P2, and PI(3,4,5)P3, respectively. These lipids bind to the pleckstrin homology (PH) domain of various proteins, including protein kinase B (PKB), otherwise known as AKT (or Akt).

AKT is a serine/threonine protein kinase that plays a key role in multiple cellular processes such as glucose metabolism, cell proliferation, apoptosis, transcription and cell migration. AKT is involved in the PI3K/AKT/mTOR pathway and other signaling pathways. AKT is directly activated by phosphorylation by its activating kinases at threonine 308 (phosphorylated by phosphoinositide dependent kinase 1; PDPK1) and at serine 473 (phosphorylated by mammalian target of rapamycin complex 2; mTORC2). Phosphorylation of both sites on AKT is necessary for activation, but phosphorylation at threonine 308 only stabilises the activation loop while the site at serine 473 is necessary for full activation (Alessi et al, (1996) EMBO J. 15 (23): 6541-51; Blume-Jensen et al, (2001) Nature 411(6835): 355-65). Activated Akt then proceeds to activate or deactivate its myriad substrates (e.g. mTOR) via its kinase activity. PI3K-dependent Akt activation can be regulated through the tumor suppressor PTEN (which is a phosphatase), activation of which significantly reduces the rate of Akt activation.

The phosphorylation of the two phosphorylation sites on AKT (which are critical for AKT activation) is inhibited by the selective PI3K inhibitor, LY294002. Akt plays a key role in protecting cells from apoptosis through interactions with diverse downstream targets including NF-kB, Bad, caspase-9, and p53 among others. Wang at al., (2002) Clin. Can. Res. 8: 1940-7 reported that LY294002 potentiated HDAC inhibitor-mediated lethality in the KM20 human colon cancer cell line in association with diminished activation of Akt. Rahmani at al., supra followed on from this work to indicate that LY294002 dramatically enhances HDAC inhibitor-mediated mitochondrial damage and apoptosis in human leukemia cells, but that these events are more closely related to interference with induction of Raf/MEK/ERK and p21^{CIP1/WAF1} than to inhibition of the Akt pathway. The HDAC inhibitors used by Rahmani et al., supra included sodium butyrate (SB), suberanoyl hydroxamic acid (SAHA; vorinostat) and MS-275. These inhibitors predominantly inhibit Class I HDACs or HDACs in general. Specific HDAC Class II inhibitors have not previously been combined with inhibitors of the PI3K pathway.

Liu at al. (2006) J. Biol. Chem. 281(42): 31359-68 reported that SAHA stimulates NE-KB transcription through a signalling cascade that involves activation of both AKT and the p300 acetyltransferase. SAHA predominantly inhibits HDAC1.

Bao at al (April 2011) Antitumor Activity of a Dual PI3K and HDAC Inhibitor in Hematologic Cancer Models: Abstract number 2615; 102^{nd} AACR Meeting, Orlando, Fla. identifies a dual PI3K and HDAC Inhibitor CUDC-907 (Curls, Inc). Bao explains that blocking PI3K can up-regulate other survival pathways which can in turn be overcome by HDAC inhibition. Synergistic effects can be achieved by inhibition of both HDAC and PI3K in cancer cells. CUDC-907 is described as inhibiting the PI3K-AKT pathway, but also suppresses other vital signalling pathways and induces apoptosis in cancer cells via epigenetic modification. Experiments show that CUDC-907 inhibits HDAC1, HDAC3, and HDAC10 with significantly greater potency than other HDAC subtypes.

Saunders et al (April 2011) Preclinical evaluation of dual PI3K-mTOR inhibitors and histone deacetylase inhibitors in head and neck squamous cell carcinoma: Abstract number 2559; 102^{nd} AACR Meeting, Orlando, Fla. describes similar synergism between inhibitors of PI3K-mTOR and inhibitors of HDAC in vitro in a model of head and neck squamous cell carcinoma (HNSCC). Saunders et al (2011) reported that that PI3K, AKT, and dual PI3K-mTOR inhibitors (LY294002, Wortmannin, AKT VIII, BEZ-235, BKM-120 and BGT-226) caused a marked in vitro enhancement of cytotoxicity induced by HDAC Inhibitors (vorinostat (SAHA), depsipeptide, valproic acid and LBH-589) in HNSCC cancer cells (SCC25, Cal27, Detroit 562). However, Saunders et al (2011) saw no evidence of improved efficacy with an HDAC Inhibitor/PI3K Inhibitor combination in vivo. The HDAC inhibitors used by Sanders et al (2011) are non-selective HDAC inhibitors, whereas valproic acid preferentially inhibits HDAC1. LBH-589 (Panabinostat; see Atadja et al (2009) Cancer Letters 280: 233-41), for example, is a pan-HDAC inhibitor that mostly inhibits HDACs 1, 2, 3 and 9 (see Khan et al (2008) Biochem. J. 409: 581-9) and is associated with a profile similar to vorinostat (SAHA).

The listing or discussion of an apparently prior-published document in this specification should not necessarily be taken as an acknowledgement that the document is part of the state of the art or is common general knowledge.

Surprisingly, the present inventors have discovered that compounds that selectively inhibit HDAC6 combine synergistically with agents that reduce AKT activity to enhance cancer cell growth inhibition and apoptosis in vitro and in vivo. Some effect was seen with pan-HDAC inhibitor SAHA, but only at much higher concentrations than with HDAC6 inhibitors. We note that HDAC6 inhibition was associated with an increase in phosphorylated AKT (P-AKT) that was not seen with SAHA. In fact, pan-HDAC inhibitors SAHA and LBH-589 are associated with a decrease in P-AKT (Chou et al, (2011) PLoS ONE 6(3); Suzuki et al, (2009) Cancer Chemother Pharmacol 64:1115-1122; Qian et al, (2006) Clin Cancer Res 2006; 12: 634-642).

Thus, in a first aspect the present invention provides a composition comprising a Histone Deacetylase 6 (HDAC6) inhibitor and an AKT inhibitor.

By “HCAC6 inhibitor” we include any compound that has the effect of preferentially reducing and/or blocking the activity of Histone Deacetelase 6, i.e. a compound that “selectively” inhibits HDAC6. It is preferred that the compound inhibits HDAC6 in preference to other classes of HDAC, such as Class I HDACs, e.g. HDAC1 and HDAC8.

For example, the compound may be over 3-fold, or over 5-fold, or over 10-fold, or over 20-fold more specific for HDAC6 inhibition than HDAC1 inhibition. By “selectively” inhibits we include the meaning that the compound has an IC₅₀ value for HDAC6 which is lower than for members of other HDACs or HDAC classes (e.g. Class I HDACs: HDAC1, HDAC8). Preferably, the compound has an IC₅₀ value at least five or ten times lower than for at least one other HDAC, and preferably more than 100 or 500 times lower. More preferably, the compound which selectively inhibits HDAC6 has an IC₅₀ value more than 1000 or 5000 times lower than for at least one other HDAC. Preferably, the at least one other HDAC is a mammalian, more preferably human, HDAC. Also preferably, the compound which selectively inhibits HDAC6 has a lower IC₅₀ value than for at least 2 or 3 or 4 or 5 or at least 10 other HDACs, as the case may be. Most preferably, the compound which selectively inhibits HDAC6 has an IC₅₀ value at least five times lower than for all other HDACs, and preferably at least 10, 50, 100 or 500 times lower.

The HDAC6 inhibitor compound may inhibit the enzymatic activity of HDAC6, or act in another way to prevent the function of HDAC6. As will be understood by a person skilled in the art, a compound that inhibits HDAC6 is generally expected to cause an increase in markers of HDAC6 inhibition and may be identified by such property. For example, it may be expected that inhibition of HDAC6 will lead to an increase in acetyl-alpha-tubulin, and/or acetyl-Hsp90, and/or acetyl-cortacin, and/or acetyl-peroxiredoxin I, and/or acetyl-peroxiredoxin II, amongst other markers of HDAC 6 inhibition. HDAC6 inhibition is also believed to be linked to the acetylation of nuclear histones in vivo (Wang et al, (2009) Cell. 138(5): 1019-1031). Therefore, HDAC6 inhibition may also be expected to lead to an increase in acetyl nuclear histones. Further, the inhibitor of HDAC6 may act to prevent or reduce the transcription, translation, post-translational processing and/or mobilisation of HDAC6 (i.e. reduce the expression of HDAC6), or an upstream activator of the expression of HDAC6. Thus, the HDAC inhibitor compounds may be, for example, small chemical entities, antibodies, small interfering RNA, double-stranded RNA or Ribozymes. Examples of small chemical entity inhibitors of HDAC6 include the mustard prodrug hydroxamic acid-based histone deacetylase inhibitors identified in WO 2008/050125, for example, HDAC-C1A or HDAC-C1B (structures provided below). Further examples of HDAC6 inhibitors include tubacin, tubastatin A, and cyclic tetrapeptide hydroxamic acids (Butler et al, (2010) J. Am. Chem. Soc., 132: 10842-10846; Haggarty et al, (2003) Proc. Natl. Acad. Sci. USA 100: 4389-4394; Jose et al, (2004) Bioorg. Med. Chem. 12: 1351-1356).

By “AKT inhibitor” we include any compound that has the effect of preferentially reducing and/or blocking the activity of AKT. The inhibitor may act directly on AKT, for example by preventing phosphorylation of AKT or de-phosphorylating AKT, for example at Ser473 and/or Thr308, or alternatively, the inhibitor may act via the inhibition of an upstream activator (or multiple activators) of AKT in the PI3K/AKT/mTOR signalling pathway or other pathway involved in apoptosis, or via the activation of a upstream inhibitor of AKT. It is preferred that the AKT inhibitor acts to reduce and/or block the activity of AKT via multiple pathways such that effective inhibition is achieved. Such a compound may, for example, act by inhibition of up-stream effectors/activators of AKT in both the PI3K pathway and the mTOR pathway. Yet further, the inhibitor of AKT may act to prevent or reduce the transcription, translation, post-translational processing and/or mobilisation of AKT (i.e. reduce the expression of AKT), or an upstream activator of the expression of AKT. Alternatively, the “AKT inhibitor” may be a compound that counteracts the survival mechanism modulated by AKT activity by acting downstream of AKT to overcome the action of increased AKT activity. For example, such a compound may induce apoptosis via a mechanism involving AKT but by acting on downstream modulators of AKT, for example, BCL-2 inhibition.

Thus, examples of “inhibitors of AKT” within the meaning of the present invention include compounds that inhibit PI3K or downstream effectors of PI3K (e.g. PI), compounds that inhibit PDPK1 and/or mTORC2 or associated kinases (e.g. PHT-427 (Meuillet, et al, (2010) Mol Cancer Ther. 9(3): 706-717); BX-795, BX-912 and BX-320 (Chung et al, (2005) Oncogene 24, 7482-7492); and PP-27 and OSI-027 (Evangelisti et al (2011), Leukemia 25, 781-791)), compounds that inhibit AKT directly (i.e. target AKT enzymatic activity) (e.g. AT7867 (Grimshaw K M et al. (2010) Mol Cancer Ther. 9(5):1100-10); KRX-0401 (perifosine) (Kondapaka et al, (2003) Mol Cancer Ther 2: 1093-1103); MK-2206 (Hirai et al. (2010) Mol Cancer Ther 9(7)), compounds that activate PTEN (e.g. Trastuzumab (Nagata et al. (2004) Cancer cell (6))) and any other compounds that lead to a reduction in AKT activation. The compounds may be, for example, small chemical entities, antibodies, small interfering RNA, double-stranded RNA (e.g. RX-0201, A (AKT anti sense)) or Ribozymes. Examples of appropriate small chemical entities include BEZ-235, PI-103 (Park et al (2008) Leukemia 22: 1698-1706), API-2, LY294002, Wortmannin, AKT VIII, BKM120, BGT226, Everolimus, Choline kinase inhibitors (e.g. CK37 (Clem et al (2011) Oncogene 1-11); H89 (Wieprecht et al. (1994) Biochem. J. 297, 241-247); MN58b and TCD828 (Tin Chua et al. (2009) Molecular Cancer, 8:131)), bcl-2 inhibitor (e.g. ABT-737), Hsp-90 inhibitors (e.g. Geldanamycin (Stebbins et al (1997) Cell. 89(2): 239-50); and derivatives of Geldanamycin, for example, 17-AAG and 17-DMAG (Hollingshead M et al. (2005) Cancer Chemother Pharmacol. August; 56 (2):115-25), multi-kinase inhibitors (e.g. sunitinib), mTOR kinase inhibitors (e.g. Temsirolimus), proteasome inhibitors (e.g. bortezomib), and TORC1/TORC2 inhibitors (e.g. Palomid 529 (P529)).

Not wishing to be bound by any theory, the present inventors believe that selective inhibition of HDAC6 on the one hand activates death pathways mediated by increased acetylation of HSP-90, alpha-tubulin, and p53, amongst others, and on the other hand activates survival pathways mediated by the increased acetylation of, and thus deactivation of, PTEN leading to increased AKT activity. Thus, inhibition of AKT with HDAC6 inhibition provides an additive killing effect on cells enhancing cytotoxicity. This is surprisingly more effective than pan-HDAC class I inhibition in combination with PI3K inhibition. Also, pan-HDAC inhibitors are associated with high toxicity. HDAC6 is less ubiquitously expressed than other HDACs. Thus, targeting HDAC6 and AKT may provide more therapeutic benefit to patients than previously suggested compositions while reducing side effects of therapy.

In a second aspect, the invention provides a pharmaceutical composition/formulation comprising the composition of the first aspect, in admixture with a pharmaceutically acceptable excipient, adjuvant, diluent or carrier.

Preferably, the composition/formulation is a unit dosage containing a daily dose or unit, daily sub-dose or an appropriate fraction thereof, of the active ingredients.

The composition of the invention will normally be administered orally or by any parenteral route, in the form of a pharmaceutical formulation comprising the active ingredients, optionally in the form of a non-toxic organic, or inorganic, acid, or base, addition salt, in a pharmaceutically acceptable dosage form. Depending upon the disorder and patient to be treated, as well as the route of administration, the compositions may be administered at varying doses.

The composition of the invention can be administered alone but will generally be administered in admixture with a suitable pharmaceutical excipient, diluent or carrier selected with regard to the intended route of administration and standard pharmaceutical practice.

For example, the composition of the invention can be administered orally, buccally or sublingually in the form of tablets, capsules, ovules, elixirs, solutions or suspensions, which may contain flavouring or colouring agents, for immediate-, delayed- or controlled-release applications. Thus, for example, the composition of the invention may be formulated with an enteric coating or film coating or other coating as appropriate to avoid or slow degradation of the composition in the stomach of the patient, as would be understood by a person of skill in the art of drug delivery technologies. Appropriate coatings to protect the composition from degradation in the stomach will be well known to the skilled person. For example, a capsule or tablet comprising the composition of the invention may be provided with an enteric coating comprising methyl acrylate-methacrylic acid copolymers, cellulose acetate succinate, hydroxy propyl methyl cellulose phthalate, hydroxy propyl methyl cellulose acetate succinate (hypromellose acetate succinate), polyvinyl acetate phthalate (PVAP), methyl methacrylate-methacrylic acid copolymers, sodium alginate and/or stearic acid, or any other appropriate coating. The composition of the invention may also be administered via intracavernosal injection.

Such tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the composition of the invention may be combined with various sweetening or flavouring agents, colouring matter or dyes, with emulsifying and/or suspending agents and with diluents such as water, ethanol, propylene glycol and glycerin, and combinations thereof.

The composition of the invention can also be administered parenterally, for example, intravenously, intra-arterially, intraperitoneally, intrathecally, intraventricularly, intra-sternally, intracranially, intra-muscularly or subcutaneously, or may be administered by infusion techniques. The compositions are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

For oral and parenteral administration to human patients, the daily dosage level of the composition of the invention, or compounds for the medical uses of the invention, will usually be from 1 to 1000 mg per adult (i.e. from about 0.015 to 15 mg/kg), administered in single or divided doses.

Thus, for example, the tablets or capsules of the composition of the invention may contain from 1 mg to 1000 mg of active compounds (i.e. HDAC6 inhibitor and AKT inhibitor) for administration singly or two or more at a time, as appropriate. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention.

The composition of the invention can also be administered intranasally or by inhalation and may be conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A3 or 1,1,1,2,3,3,3-heptafluoropropane (HFA 227EA3), carbon dioxide or other suitable gas. In the case of a pressurised aerosol, the dosage unit may be determined by providing a valve to deliver a metered amount. The pressurised container, pump, spray or nebuliser may contain a solution or suspension of the active compounds, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of a compound of the invention and a suitable powder base such as lactose or starch.

Aerosol or dry powder formulations are preferably arranged so that each metered dose or “puff” contains at least 1 mg of the composition of the invention for delivery to the patient. It will be appreciated that the overall daily dose with an aerosol will vary from patient to patient, and may be administered in a single dose or, more usually, in divided doses throughout the day.

Alternatively, the composition of the invention can be administered in the form of a suppository or pessary, or may be applied topically in the form of a lotion, solution, cream, ointment or dusting powder. The composition of the invention may also be transdermally administered, for example, by the use of a skin patch. The composition may also be administered by the ocular route, particularly for treating diseases of the eye.

For ophthalmic use, the composition of the invention can be formulated as a micronised suspension in isotonic, pH adjusted, sterile saline, or, preferably, as a solution in isotonic, pH adjusted, sterile saline, optionally in combination with a preservative such as a benzylalkonium chloride. Alternatively, the composition may be formulated in an ointment such as petrolatum.

For application topically to the skin, the composition of the invention can be formulated as a suitable ointment containing the active compounds suspended or dissolved in, for example, a mixture with one or more of the following: mineral oil, liquid petrolatum, white petrolatum, propylene glycol, polyoxyethylene polyoxypropylene compound, emulsifying wax and water. Alternatively, the composition can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

Formulations suitable for topical administration in the mouth include lozenges comprising the active ingredients in a flavoured basis, usually sucrose and acacia or tragacanth; pastilles comprising the active ingredients in an inert basis such as gelatin and glycerin, or sucrose and acacia; and mouth-washes comprising the active ingredients in a suitable liquid carrier.

Generally, in humans, oral or topical administration of the composition of the invention is the preferred route, being the most convenient. In circumstances where the recipient suffers from a swallowing disorder or from impairment of drug absorption after oral administration, the composition may be administered parenterally, e.g. sublingually or buccally.

The compounds of the compositions of the invention may be further presented in the form of “prodrugs”. The term “prodrug” as used in this application refers to a precursor or derivative form of a pharmaceutically active substance that is less cytotoxic to tumour cells compared to the parent drug and is capable of being enzymatically activated or converted into the more active parent form (see, for example, D. E. V. Wilman “Prodrugs in Cancer Chemotherapy” Biochemical Society Transactions 14, 375-382 (615th Meeting, Belfast 1986) and V. J. Stella et al. “Prodrugs: A Chemical Approach to Targeted Drug Delivery” Directed Drug Delivery R. Borchardt et al. (ed.) pages 247-267 (Humana Press 1985)).

In a third aspect, the invention provides a kit of parts comprising a Histone Deacetylase 6 (HDAC6) inhibitor, and an AKT inhibitor.

It is envisaged that the kit may be provided with instructions for administration of the components according to the regimes provided herein. The components of the kit may be presented as pharmaceutical formulations comprising the components in admixture with a pharmaceutically acceptable excipient, adjuvant, diluent or carrier, as described above in relation to the second aspect. Thus, the kit may comprise a pharmaceutical formulation of an HDAC6 inhibitor, in conjunction with a pharmaceutical formulation of an AKT inhibitor.

In an embodiment of any of the preceding aspects, it is envisaged that the composition, pharmaceutical composition or kit of parts may further comprise one or more anti-cancer agents.

By “anti-cancer agent” we include any compounds known to be toxic to cancer cells, for example cancer chemotherapy agents. Such compounds have preferably been shown to be useful in the treatment of cancer. Examples of such anti-cancer agents include tyrosine kinase inhibitors (imatinib, gefitinib, and others); DNA methyltransferase inhibitors (5-aza-2′-deoxycytidine, 5-azacytidine, and others); tamoxifen; aromatase inhibitors (anastrozole, letrozole, exemestane, and others); fulvestrant progestogens (megestrol acetate, medroxyprogesterone acetate, gestonorone caproate, norethisterone, and others); anti-androgens (cyproterone acetate, flutamide, bicalutamide, and others); luteinising hormone releasing hormone analogues (goserelin, leuprorelin, buserelin, and others); oestrogens (ethinylestradiol, diethylstilbestrol, and others); anthracyclines (doxorubicin, epirubicin, daunorubicin, idarubicin, aclarubicin, and others); topoisomerase I inhibitors (etoposide, teniposide, and others); topoisomerase II inhibitors (irinotecan, topotecan, and others); fluoropyrimidines (5-fluorouracil, FUdR, tegafur, capecitabine, gemcitabine, raltitrexed, and others); alkylating agents (cyclo-phosphamide, ifosfamide, chlorambucil, thiotepa, busulfan, carmustine, mustine, estramustine, lomustine, treosulfan, melphalan, dacarbazine, procarbazine, and others); methotrexate; hydroxyurea; platinum compounds (cisplatin, carboplatin, oxaliplatin, and others); taxanes (paclitaxel, docetaxel, and others); purine analogues (mercaptopurine, pentostatin, cytarabine, fludarabine, thioguanine, cladribine, and others); vinca alkaloids (vincristine, vinblastine, vinorelbine, vindesine, and others); proteasome inhibitors (bortezomib, lactacystin, MG-132, and others); retinoids (ATRA, bexarotene, tretinoin, isotretinoin, and others); immunosuppressant and immunomodulating drugs (azathioprine, tacrolimus, ciclosporin, and others), radiation therapy, and Iodine 123 radio labeled Meta-iodobenzylguanidine (MIBG).

Thus, in the preceding embodiment, the anti-cancer agent(s) may be selected from the group comprising, but not limited to, apoptosis inducing drugs (e.g. drugs that target bax and bcl-2), chemotherapy agents (e.g. taxanes), biologic therapies (e.g. antibodies that target estrogens or androgens), proteasome inhibitors (e.g. bortezomib), and HSP90 inhibitors (e.g. 17-AAG).

In a further aspect, the present invention provides a composition, pharmaceutical composition or kit of parts as defined herein for use in medicine.

The compositions of the invention may be of use in the prevention or treatment of a range of conditions or diseases in which inhibition of HDAC6 in combination with inhibition of AKT may prevent, inhibit, or ameliorate the pathology and/or symptomatology of the condition or disease, enhance or otherwise augment the activity of any other agent used to prevent or treat the condition or disease; sensitise the condition or disease to any preventive or therapeutic agent. It is envisaged that the condition or disease may be caused by or associated with abnormal cell proliferation. Thus, the compositions of the invention may be particularly useful in the treatment of any disease where stimulation of apoptosis may be beneficial.

For example, the compositions of the invention may be of clinical use in the prevention or treatment of cancer. The compositions of the invention may also be used to prevent or treat premalignant haematological conditions e.g. myelodysplasia and myelodysplastic syndromes. The compositions of the invention may also be used to prevent or treat haemoglobinopathies, e.g. sickle cell anaemia and β-thalassaemia. The composition of the invention may also be used to prevent or treat microbial infections e.g. superficial and invasive fungal infections (Candida sp., Aspergillus sp., coccidioidomycosis, histoplasmosis and others), or parasitic infections e.g. malaria (Plasmodium vivax and P. falciparum) or other protozoal infections e.g. Pneumocystis carinii; Toxoplasma gondii.

The compositions of the invention may also be used to prevent or treat neurodegenerative diseases, both inherited (e.g. Huntington's disease) and acquired (e.g. Alzheimer's disease) (further examples of appropriate neurodegenerative diseases include Rubinstein-Taybi syndrome, Rett syndrome, and Friedreich's ataxia), hyperproliferative diseases (e.g. keloid, psoriasis hypertrophic cardiomyopathy, hepatic and biliary fibrosis) or connective tissue diseases (e.g. systemic lupus erythematosus), neovascular diseases, e.g. of the eye (e.g. diabetic retinopathy, neovascular glaucoma, corneal neovascularisation), diabetes mellitus and multiple sclerosis.

The compositions of the invention may also be used to prevent or treat graft or stent occlusion (e.g. stents impregnated with the compositions of the invention, or coronary artery bypass grafts), as chemoprevention in high risk groups (e.g. cancer associated with familial polyposis coli, ulcerative colitis or BRCA1 or BRCA2 gene mutations) or in the prevention of premature labour and parturition.

The compositions of the invention may also be used to prevent or overcome drug resistance e.g. imatinib in Philadelphia chromosome positive chronic myelogenous myeloid leukaemia; anthracyclines and other cytotoxic chemotherapy drugs; endocrine therapies in hormone responsive cancers (tamoxifen and aromatase inhibitors and fulvestrant in breast cancer, anti-androgens and luteinising hormone releasing hormone analogues in prostate cancer).

Thus, in a yet further aspect, the invention provides a composition, pharmaceutical composition or kit of parts as defined herein for use in the prevention or treatment of cancer.

Accordingly, in a further aspect, the invention provides a composition, pharmaceutical composition or kit of parts as defined herein for use in the prevention or treatment of neuro-degenerative conditions, including, but not limited to, Alzheimer's disease, Parkinson's disease, Huntington's disease, Amyotrophic lateral sclerosis (ALS), spinal and bulbar muscular atrophy (SbMA), Rubinstein-Taybi syndrome, Rett syndrome, and Friedreich's ataxia, or autoimmune diseases, including Rheumatoid Arthritis, Myasthenia Gravis, and Multiple Sclerosis.

The invention further provides a Histone Deacetylase 6 (HDAC6) inhibitor for use in the prevention or treatment of cancer, a neuro-degenerative condition, and/or an autoimmune disease, in a patient who has been administered an AKT inhibitor. It is envisaged that the patient will have been administered the AKT inhibitor shortly before administration of the HDAC6 inhibitor, for example, immediately before administration, or up to an hour after administration, or according to the administration regimes provided herein.

On the other hand, the invention also provides an AKT inhibitor for use in the prevention or treatment of cancer, a neuro-degenerative condition, and/or an autoimmune disease, in a patient who has been administered a Histone Deacetylase 6 (HDAC6) inhibitor. Again, it is envisaged that the patient will have been administered the HDAC6 inhibitor shortly before administration of the AKT inhibitor, for example, immediately before administration, or up to an hour after administration, or according to the administration regimes provided herein.

The invention further provides for the use of a composition, pharmaceutical composition or kit of parts as defined herein in the manufacture of a medicament for preventing or treating cancer, a neuro-degenerative condition, and/or an autoimmune disease. The Histone Deacetylase 6 (HDAC6) inhibitor and the AKT inhibitor may be provided for administration concurrently or for administration sequentially. Further, the Histone Deacetylase 6 (HDAC6) inhibitor and the AKT inhibitor may be provided for administration concurrently or sequentially with the one or more anti-cancer agents described herein in relation to other aspects.

In a yet further aspect, the invention provides a method of preventing or treating cancer, a neuro-degenerative condition, and/or an autoimmune disease, comprising the step of administering a Histone Deacetylase 6 (HDAC6) inhibitor and an AKT inhibitor to a patient in need thereof. The Histone Deacetylase 6 (HDAC6) inhibitor and the AKT inhibitor may be administered concurrently or sequentially in the methods of the invention. Further, the Histone Deacetylase 6 (HDAC6) inhibitor and the AKT inhibitor may be administered concurrently or sequentially in the methods of the invention with one or more anti-cancer agents as described in relation to other aspects.

By “cancer” we include both tumorous and non-tumorous cancers. We include primary and/or locally advanced and/or metastatic solid tumours, including but not limited to cancers of the breast, upper aerodigestive tract, endocrine system including thyroid; adrenal gland, parathyroid, carcinoid and pancreatic neuroendocrine tumours, lung, oesophagus, stomach, pancreas, liver, gall bladder and hepatobiliary system, small intestine, colorectal, ovarian, bladder, prostate, gynaecologic tumours (vulva, vagina, cervix, endometrium, uterine, fallopian tubes), testis, penis and urethra, renal, central nervous system, skin (basal and squamous cell carcinomas and melanoma), sarcomas of soft tissue and bone, mesothelioma, developmental cancers such as testicular or ovarian germ cell tumours and teratomas, and gestational trophoblastic tumours including hydatidiform moles and choriocarcinomas, primary and metastatic bone tumours, intraocular melanomas, and solid tumours of childhood. We also include haematological malignancies including but not limited to acute and chronic leukaemias (acute myelogenous leukaemia, acute lymphoblastic leukaemia, acute promyelocytic leukaemia, chronic myelogenous leukaemia, chronic lymphocytic leukaemia, hairy cell leukaemia) lymphomas (Hodgkin's disease and non-Hodgkin's lymphomas, cutaneous and peripheral T-cell lymphomas), and plasma cell tumours including multiple myeloma. The cancer may be a tumorous or non-tumorous cancer.

Thus, in any of the aspects and embodiments of the invention, the cancer may be selected from, but not limited to, the group comprising breast cancer, ovarian cancer, prostate cancer, bowel cancer, lung cancer, neuroblastoma, leukaemia, lymphoma and/or melanoma. It will be understood that the invention may be appropriate for use in the prevention and/or treatment of any cancer or disease characterised by inappropriate cell growth, where apoptosis of cells will be desirable.

It is intended that the pharmaceutical compositions and inhibitors of the invention may be used in combination with appropriate targeting means and/or delivery means to aid in targeting and delivery of the medicaments to the appropriate region, organ, tissue and/or cell in the patient to be treated. Thus, such targeting means may aid in improving efficacy of the treatment and in reducing unwanted side effects of the treatment. Appropriate targeting means include antibodies or antibody fragments or derivatives thereof that specifically recognise target tissue/cells. Alternative targeting means may include virus particles, for example adenovirus particles or retrovirus particles that are modified to target, or naturally target, the target tissue/cells.

In an embodiment of the kit, HDAC6 inhibitor, AKT inhibitor, use or method described herein, the HDAC6 inhibitor may be for administration, or may be administered, to the patient up to 24 hours after administration of the AKT inhibitor; for example up to 8 hours after administration of the AKT inhibitor, or for example up to 1 hour after administration of the AKT inhibitor. For example, the HDAC6 inhibitor may be for administration, or may be administered, to the patient immediately after up to 48 hours after administration of the AKT inhibitor, or between 1 minute and 36 hours, or between 5 minutes and 30 hours, or between 10 minutes and 24 hours, or between 15 minutes and 20 hours, or between 20 minutes and 15 hours, or between 25 minutes and 10 hours, or between 30 minutes and 9 hours, or between 35 minutes and 8 hours, or between 40 minutes and 7 hours, or between 45 minutes and 6 hours, or between 50 minutes and 5 hours, or between 55 minutes and 4 hours, or between 1 and 3 hours, or between 1 and 2 hours, or any combination thereof, after administration of the AKT inhibitor. It is intended that the HDAC6 inhibitor should be administered at a time when tissue AKT inhibition is still be detectable in the patient. Appropriate timing will be determined for each drug. It will be appreciated that the timing of such administration will depend to a great extent on the pharmacodynamics and pharmacokinetics of the AKT inhibitor. Nevertheless, the above regimes provide examples of appropriate administration times.

In an embodiment of the kit, HDAC6 inhibitor, AKT inhibitor, use or method described herein, the AKT inhibitor may be for administration, or may be administered, to the patient up to 24 hours after administration of the HDAC6 inhibitor; for example up to 8 hours after administration of the HDAC6 inhibitor, or for example up to 1 hour after administration of the HDAC6 inhibitor. For example, the AKT inhibitor may be for administration, or may be administered, to the patient immediately after up to 48 hours after administration of the HDAC6 inhibitor, or between 1 minute and 36 hours, or between 5 minutes and 30 hours, or between 10 minutes and 24 hours, or between 15 minutes and 20 hours, or between 20 minutes and 15 hours, or between 25 minutes and 10 hours, or between 30 minutes and 9 hours, or between 35 minutes and 8 hours, or between 40 minutes and 7 hours, or between 45 minutes and 6 hours, or between 50 minutes and 5 hours, or between 55 minutes and 4 hours, or between 1 and 3 hours, or between 1 and 2 hours, or any combination thereof, after administration of the HDAC6 inhibitor. It is intended that the AKT inhibitor should be administered at a time when tissue HDAC6 inhibition is still be detectable in the patient. Appropriate timing will be determined for each drug. It will be appreciated that the timing of such administration will depend to a great extent on the pharmacodynamics and pharmacokinetics of the HDAC6 inhibitor. Nevertheless, the above regimes provide examples of appropriate administration times.

In any aspect of the invention, the HDAC6 inhibitor may be a compound of formula I, or a compound of formula IX; wherein formula I is

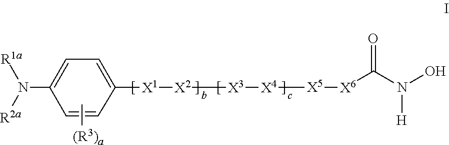

wherein R^{1a} represents C₁₋₄ alkyl (which latter group is optionally substituted by one or more substituents selected from halogeno and aryl), aryl, (CH₂)₂-L¹ or the structural fragment

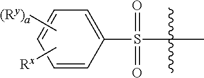

wherein R^{x} represents H or N(R^{1b})R^{2b}; R^{1b} and R^{2b} independently represent C₁₋₄ alkyl (which latter group is optionally substituted by one or more substituents selected from halogeno and aryl), aryl or (CH₂)₂-L²; R^{y} represents halogeno or C₁₋₄ alkyl; R^{2a} represents H, C₁₋₄ alkyl (which latter group is optionally substituted by one or more substituents selected from halogeno and aryl), aryl or (CH₂)₂-L³; L¹, L² and L³ each represents, independently at each occurrence, a leaving group; R³ represents halogeno or C₁₋₄ alkyl; a represents, independently at each occurrence, an integer from 0 to 4; X¹-X² represents C(O)—CH(Y¹), C(H)═C(Y¹), CH₂—CH(Y¹), NH—CH(Y¹), CH₂—C(O), NH—C(O) or CH(Y¹); b represents 0 or 1; X³-X⁴ represents CH═C(Y²), O—CH(Y²), NH—CH(Y²), O—C(O) or NH—C(O); c represents an integer from 0 to 10; X⁵-X⁶ represents CH₂—CH₂, CH═CH or O—CH₂; and Y¹ and Y² independently represent, at each occurrence, H or C₁₋₄ alkyl; or a pharmaceutically acceptable derivative thereof, provided that at least one of the following is the case: (a) R^{1a} represents (CH₂)₂-L¹; (b) R^{1b} and/or R^{2b} represents (CH₂)₂-L²; (c) R² represents (CH₂)₂-L³; and wherein formula IX is

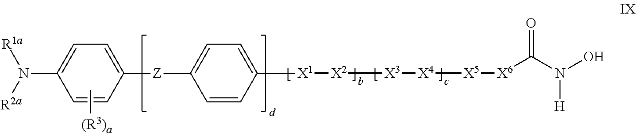

wherein R^{1a} represents C₁₋₄ alkyl (which latter group is optionally substituted by one or more substituents selected from halogeno and aryl), aryl or (CH₂)₂-L¹; R^{2a} represents H, C₁₋₄ alkyl (which latter group is optionally substituted by one or more substituents selected from halogeno and aryl), aryl or (CH₂)₂-L³; L¹, L² and L³ each represents, independently at each occurrence, a leaving group; R³ represents halogeno or C₁₋₄ alkyl; a represents, independently at each occurrence, an integer from 0 to 4; X¹-X² represents C(O)—CH(Y¹), C(H)═C(Y¹), CH₂—CH(Y¹), NH—CH(Y¹), CH₂—C(O), NH—C(O) or CH(Y¹); b represents 0 or 1; X³-X⁴ represents CH═C(Y²), O—CH(Y²), NH—CH(Y²), O—C(O) or NH—C(O); represents an integer from 0 to 10; Z represents —SO₂.NH— or —NH.SO₂—; d represents 0 or 1; X⁵-X⁶ represents CH₂—CH₂, CH═CH or O—CH₂; and Y¹ and Y² independently represent, at each occurrence, H or C₁₋₄ alkyl; or a pharmaceutically acceptable derivative thereof, provided that at least one of the following is the case: (a) R^{1a} represents (CH₂)₂-L¹; (c) R^{2a} represents (CH₂)₂-L³.

The term “leaving group”, when used herein, includes references to halogeno (e.g. Cl, Br, I) and OS(O)₂R⁴ groups wherein R⁴ is C₁₋₈ alkyl (optionally substituted by one or more fluoro atoms) or aryl (optionally substituted by one or more substituents selected from C₁₋₄ alkyl, C₁₋₄ alkoxy, NO₂ and halogeno).

Unless otherwise specified, alkyl groups and alkoxy groups as defined herein may be straight-chain or, when there is a sufficient number (i.e. a minimum of three) of carbon atoms be branched-chain, and/or cyclic. Further, when there is a sufficient number (i.e. a minimum of four) of carbon atoms, such alkyl and alkoxy groups may also be part cyclic/acyclic. Such alkyl and alkoxy groups may also be saturated or, when there is a sufficient number (i.e. a minimum of two) of carbon atoms, be unsaturated and/or interrupted by one or more oxygen and/or sulfur atoms. Unless otherwise specified, alkyl and alkoxy groups may also be substituted by one or more halogeno, and especially fluoro, atoms.

When used herein, the term “aryl” includes references to C₆₋₁₀ aryl groups such as phenyl, naphthyl and the like. Unless otherwise specified, aryl groups are optionally substituted by one or more substituents selected from halogeno, C₁₋₄ alkyl and C₁₋₄ alkoxy. When substituted, aryl groups are preferably substituted by one to five (e.g. one to three) substituents.

Pharmaceutically acceptable derivatives include salts and solvates. Salts which may be mentioned include acid addition salts.

Further, L¹ or L² may represent, independently at each occurrence, a halogeno group or OS(O)₂R⁴, wherein R⁴ is C₁₋₈ alkyl (optionally substituted by one or more fluoro atoms) or aryl (optionally substituted by one or more substituents selected from C₁₋₄ alkyl, C₁₋₄ alkoxy, NO₂ and halogeno). For example, L¹ or L² may represent, independently at each occurrence, Cl, Br, I or CH₃SO₂O (mesyloxy).

Further, R^{1a} may represent (CH₂)₂-L¹ or the structural fragment

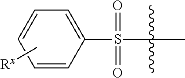

wherein R^{x} is as defined above. Further, Rx may represent N(R^{1b})R^{2b} attached in the 4-position relative to the S(O)₂ moiety.

Yet further, R^{1b} and R^{2b} may both represent (CH₂)₂-L². Further, R² may represent (CH₂)₂-L³ or, when R^{x} represents N(R^{1b})R^{2b}, then R² represents H. Further, a may represent 0. Yet further, X¹-X² may represent C(O)—CH(Y¹), C(H)═C(Y¹) or NH—C(O). Further, b may represent 1. Yet further, X³-X⁴ may represent CH═C(Y²). Further, c may represent an integer from 0 to 3. Yet further, X⁵-X⁶ may represent CH═CH or O—CH₂. Further, Y¹ and Y² may independently represent, at each occurrence, H or C₁₋₂ alkyl.

Thus, in embodiments of the invention, the HDAC6 inhibitor may be a compound of formula Ia, Ib, Ib′, Ic, Ic′, Id or Ie:

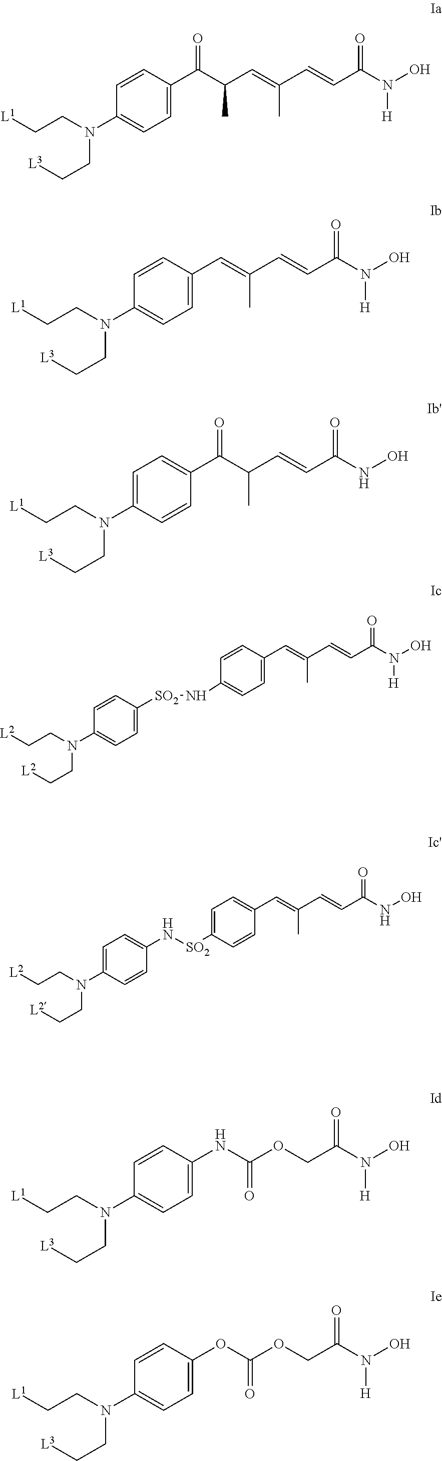

wherein L¹, L² and L³ are as defined above and L^{2′} represents a further leaving group, defined as for L² above.

In a further embodiment of the invention, the HDAC6 inhibitor may have the structure:

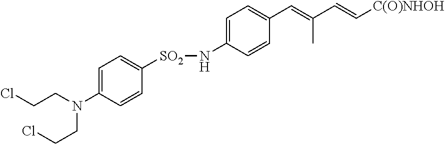

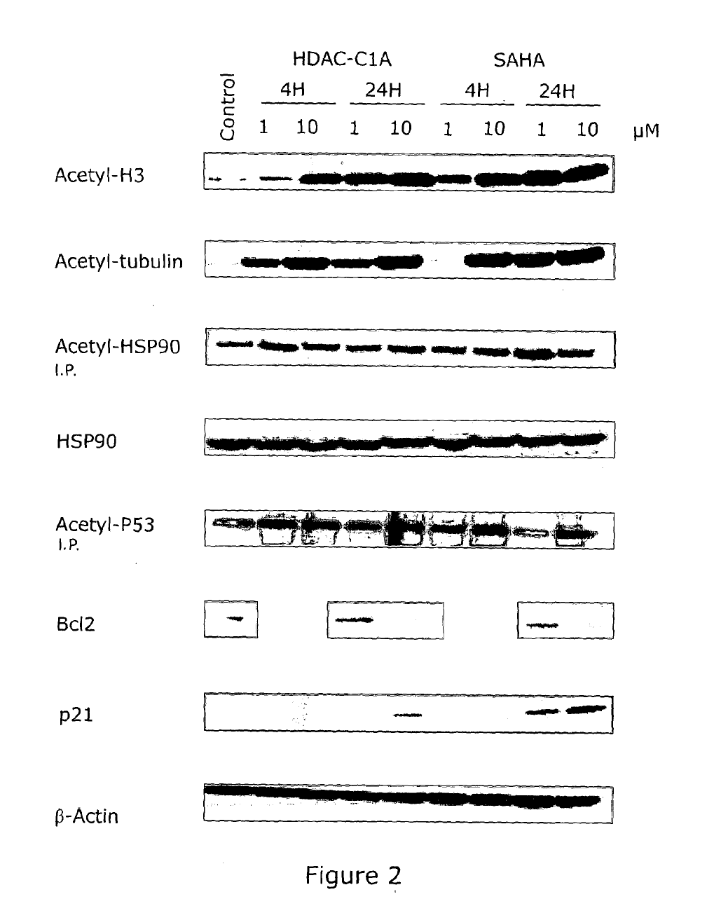

As can be seen from WO 2008/050125, the structure displayed immediately above is that of HDAC-C1A. HDAC-C1A is a particularly preferred HDAC6 inhibitor of the invention. As can be seen from the data provided herein, HDAC-C1A has particularly beneficial properties for use in the present invention. HDAC-C1A is approximately 7-fold more specific for HDAC6 than for HDAC-8 and approximately 17-fold more specific for HDAC6 when compared with HDAC1 (see Table 1). This specificity is sufficient to observe that at low concentrations and at early time point (1 μM and 4 hours) it is associated with an increase of acetyl-tubulin and other markers of HDAC6 inhibition as opposed to an increase of acetyl —H3 (a marker of class I HDAC inhibition) (see FIG. 2). The opposite is seen with SAHA, a Pan-HDAC inhibitor that mostly inhibits HDAC-1 (see Kahn et al (2008) Biochem. J. 409: 581-9) with only 1.31-fold specificity over HDAC6. Further, HDAC-C1A demonstrates properties that suggest that it will be particularly useful for delivery to central nervous system (CNS) cells. HDAC1A was shown to be a non substrate of the ABC transporters and to be associated with relatively good permeability across caco-2 cells. This suggests that HDAC-C1A will demonstrate the ability to cross the blood brain barrier: a prerequisite for reaching the CNS cells. As brain permeability remains a major limitation for drug treatment of CNS disorders, HDAC-C1A, or compounds derived from HDAC-C1A, appear to be very promising drug candidates.

In an alternative embodiment of the invention, the HDAC6 inhibitor may have the structure:

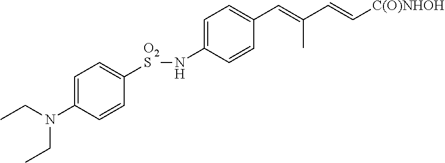

As can be seen from WO 2008/050125, the structure displayed immediately above is that of HDAC-C1B. HDAC-C1B also appears to be a promising drug candidate. In our experiments to test cytotoxicity of compounds of the invention, HDAC-C1B has demonstrated approximately 1.5-fold higher potency than HDAC-C1A (data not shown).

Compounds of formula I (including compounds of formulae Ia, Ib, Ib′, Ic, Ic′ or Id) may be made in accordance with techniques well known to those skilled in the art, for example as described in WO 2008/050125 (incorporated herein by reference in its entirety).

Alternatively, in the composition, pharmaceutical composition, kit, inhibitor, use or method of the invention, the HDAC6 inhibitor may be selected from, but not limited to, the group comprising Tubacin, Tubastatin A, and cyclic tetrapeptide hydroxamic acids.

In an embodiment of the composition, pharmaceutical composition, kit, inhibitor, use or method of the invention, the AKT inhibitor may be selected from, but not limited to, the group comprising BEZ-235, PI-103, API-2, LY294002, Wortmannin, AKT VIII, BKM120, BGT226, Everolimus, Choline kinase inhibitors, bcl-2 inhibitor (e.g. ABT-737), Hsp-90 inhibitors, multi-kinase inhibitors (e.g. sunitinib), mTOR kinase inhibitors, proteasome inhibitors (e.g. bortezomib), and TORC1/TORC2 inhibitors (e.g. Palomid 529 (P529)).

It is envisaged that the AKT inhibitor of the invention reduces AKT phosphorylation. This may be by directly interacting with AKT to prevent phosphorylation or de-phopsphorylate AKT, or alternatively could be by an indirect route, as explained above. Nevertheless, it is intended that the AKT inhibitor leads to a net reduction in AKT phosphorylation, but not necessarily a complete ablation of phosphorylation, as would be understood by the skilled person. There may be some detectable phosphorylation of AKT following use of the AKT inhibitor according to the invention, while still achieving the effects of the invention.

Experiments described in the Examples show that specific inhibition of HDAC6 using small interfering RNA (siRNA) molecules leads to an increase in the levels of phosphorylated AKT.

In an embodiment of the invention, the AKT inhibitor reduces AKT expression. Thus, it may act to prevent or reduce the transcription, translation, post-translational processing and/or mobilisation of AKT, as explained above.

In an embodiment of the invention, the HDAC6 inhibitor reduces HDAC6 expression. Thus, it may act to prevent or reduce the transcription, translation, post-translational processing and/or mobilisation of HDAC6, as explained above.

Various methods are available to reduce expression of specific genes including RNAi, antisense and triplet-forming oligonucleotides, and ribozymes.

Accordingly, such AKT or HDAC6 inhibitor may be selected from, but not limited to, the group comprising an AKT or HDAC6 specific siRNA molecule, an AKT or HDAC6 specific antisense oligonucleotide or an AKT or HDAC6 specific ribozyme.

RNAi is the process of sequence-specific post-transcriptional gene silencing in animals initiated by double stranded RNA (dsRNA) that is homologous in sequence to the silenced gene (siRNA; Hannon et al., Nature, 418 (6894): 244-51 (2002); Brummelkamp et al., Science 21, 21 (2002); and Sui et al., Proc. Natl. Acad. Sci. USA 99, 5515-5520 (2002)). The mediators of sequence-specific mRNA degradation are typically 21- and 22-nucleotide small interfering RNAs (siRNAs) which, in vivo, may be generated by ribonuclease III cleavage from longer dsRNAs. 21-nucleotide siRNA duplexes have been shown to specifically suppress expression of both endogenous and heterologous genes (Elbashir et al (2001) Nature 411: 494-498). In mammalian cells it is believed that the siRNA has to be comprised of two complementary 21mers since longer double-stranded RNAs (dsRNAs) will activate PKR (dsRNA-dependent protein kinase) and inhibit overall protein synthesis.

Duplex siRNA molecules selective for AKT or HDAC6 can readily be designed by reference to its cDNA sequence. Examples of Genbank accession numbers for HDAC6, AKT1 and AKT2 are BC069243.1, P31749, and P31751 respectively. Typically, the first 21-mer sequence that begins with an AA dinucleotide which is at least 120 nucleotides downstream from the initiator methionine codon is selected. The RNA sequence perfectly complementary to this becomes the first RNA oligonucleotide. The second RNA sequence should be perfectly complementary to the first 19 residues of the first, with an additional UU dinucleotide at its 3′ end. Once designed, the synthetic RNA molecules can be synthesised using methods well known in the art.

Antisense oligonucleotides are single-stranded nucleic acids, which can specifically bind to a complementary nucleic acid sequence. By binding to the appropriate target sequence, an RNA-RNA, a DNA-DNA, or RNA-DNA duplex is formed. The term “antisense” relates to the fact that they are complementary to the sense or coding strand of the gene. Recently, formation of a triple helix has proven possible where the oligonucleotide is bound to a DNA duplex. It was demonstrated that oligonucleotides could recognise sequences in the major groove of the DNA double helix to form a triple helix. This suggests that it is possible to synthesise a sequence-specific molecule which specifically binds double-stranded DNA via recognition of major groove hydrogen binding sites.

By binding to the target nucleic acid, antisense oligonucleotides can inhibit the function of the target nucleic acid. This may be a result of blocking the transcription, processing, poly(A)addition, replication, translation, or promoting inhibitory mechanisms of the cells, such as promoting RNA degradation.

Typically, antisense oligonucleotides are 15 to 35 bases in length. For example, 20-mer oligonucleotides have been shown to inhibit the expression of the epidermal growth factor receptor mRNA (Witters et al, Breast Cancer Res Treat 53:41-50 (1999)) and 25-mer oligonucleotides have been shown to decrease the expression of adrenocorticotropic hormone by greater than 90% (Frankel et al, J Neurosurg 91:261-7 (1999)). However, it is appreciated that it may be desirable to use oligonucleotides with lengths outside this range, for example 10, 11, 12, 13, or 14 bases, or 36, 37, 38, 39 or 40 bases. Antisense oligonucleotides specific for AKT or HDAC6 can be designed by reference to the AKT or HDAC6 cDNA sequence defined above using techniques well known in the art.

Ribozymes are RNA molecules capable of cleaving targeted RNA or DNA. Examples of ribozymes are described in, for example, Cech and Herschlag “Site-specific cleavage of single stranded DNA” U.S. Pat. No. 5,180,818; Altman et al “Cleavage of targeted RNA by RNAse P” U.S. Pat. No. 5,168,053; Cantin et al Ribozyme cleavage of HIV-1 RNA″ U.S. Pat. No. 5,149,796; Cech et al “RNA ribozyme restriction endoribonucleases and methods”, U.S. Pat. No. 5,116,742; Been et al “RNA ribozyme polymerases, dephosphorylases, restriction endonucleases and methods”, U.S. Pat. No. 5,093,246; and Been et al “RNA ribozyme polymerases, dephosphorylases, restriction endoribonucleases and methods; cleaves single-stranded RNA at specific site by transesterification”, U.S. Pat. No. 4,987,071, all incorporated herein by reference. Ribozymes specific for AKT or HDAC6 can be designed by reference to the AKT or HDAC6 cDNA sequence defined above using techniques well known in the art.

In another embodiment of the invention, the AKT inhibitor may be a compound or composition that specifically binds AKT protein. Further, it is envisaged that the AKT inhibitor may be a compound or composition that specifically binds phosphorylated AKT protein. Thus, the target site where the inhibitor binds on AKT may be phosphorylated or non-phosphorylated Ser473 and/or phosphorylated or non-phosphorylated Thr308.

Similarly, in an embodiment of the invention, the HDAC6 inhibitor may be a compound or composition that specifically binds HDAC6 protein.

The inhibitor that binds AKT protein or HDAC6 protein may be selected from, but not limited to, the group comprising an antibody, antibody fragment or derivative thereof or, in the case of the AKT inhibitor, an anti-phosphorylated AKT antibody, antibody fragment or derivative thereof. Such an inhibitor may be considered to be a neutralising antibody, antibody fragment or derivative thereof. It is envisaged that a “neutralising” antibody, antibody fragment or derivative thereof may act to prevent the enzymatic activity of AKT or HDAC6, for example by binding at the active site of AKT or HDAC6, as would be understood by a person skilled in the art.

The term “antibody” includes but is not limited to polyclonal, monoclonal, chimaeric, single chain, Fab fragments and fragments produced by a Fab expression library. Such fragments include fragments of whole antibodies which retain their binding activity for a target substance, Fv, F(ab′) and F(ab′)2 fragments, as well as single chain antibodies (scFv), fusion proteins and other synthetic proteins which comprise the antigen-binding site of the antibody. Furthermore, the antibodies and fragments thereof may be humanised antibodies, which are well known in the art.

Preferably, the patient is a human. Thus, the pharmaceutical compositions are appropriate formulated for administration to a human. Alternatively, the patient may be an animal, for example a domesticated animal (for example a dog or cat), laboratory animal (for example laboratory rodent, mouse, rat or rabbit) or an animal important in agriculture (i.e. livestock), for example, cattle, sheep, horses or goats.

Any publications referred to herein are hereby incorporated by reference in their entirety.

The invention will now be described in more detail by reference to the following non-limiting Figures and Examples.

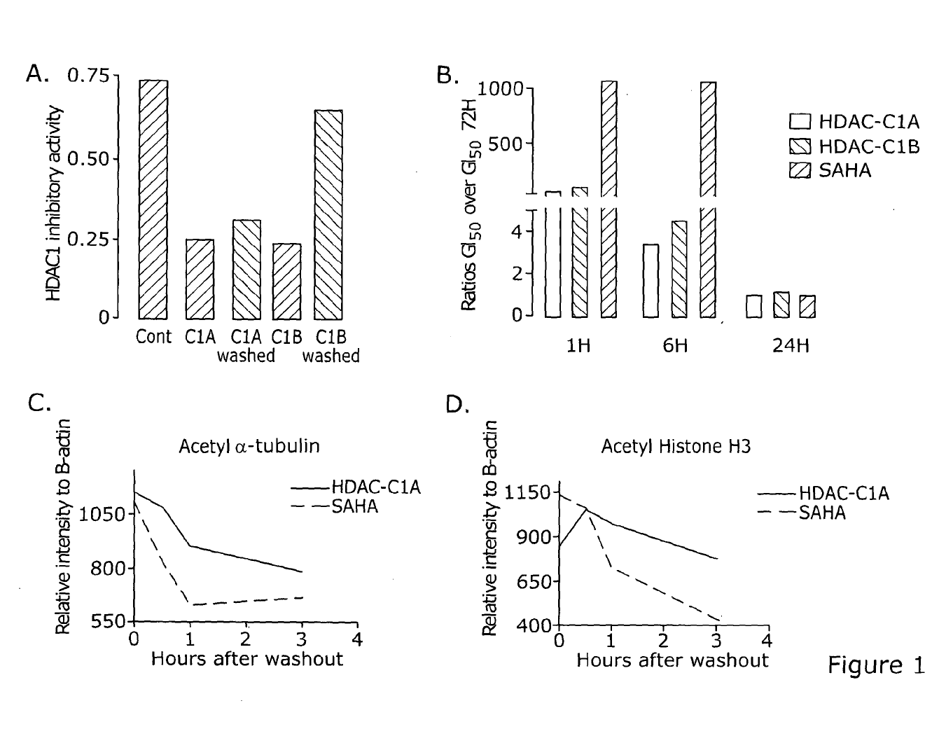

FIG. 1. HDAC-C1A irreversible activity. A. HDAC-11 inhibitory activity of HDAC-C1A was tested using HeLa nuclear extracts and compared to the negative (reversible) control HDAC-C1B. B Growth inhibitory effect of the different compounds in HCT-116 cells at indicated time and compared to growth inhibitory after 72 hours of incubation. C and D. Levels of acetyl α-tubulin and acetyl histone H3, as determined by western blotting following treatment with HDAC-C1A and SAHA for 4 hours and subsequent washout in HCT116 cells.

FIG. 2. Biomarkers screening following treatment of HCT116 cells with HDAC-C1A and SAHA.

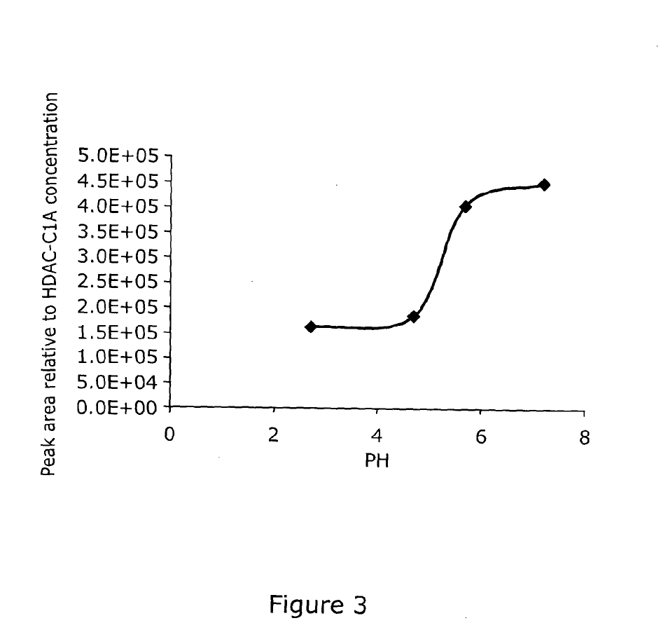

FIG. 3. Impact of the pH on the stability of HDAC-C1A. HDAC-C1A was incubated at 10 μM in PBS for 1 hour at 37° C. and the concentration was evaluated using HPLC-UV. The pH of PBS was adjusted using HCL.

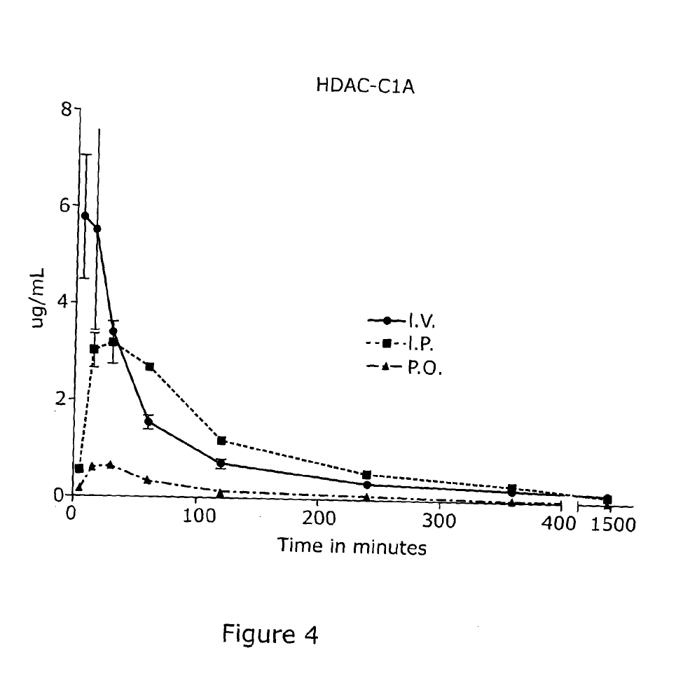

FIG. 4. Pharmacokinetic profile of HDAC-C1A following a single injection at 20 mg/kg.

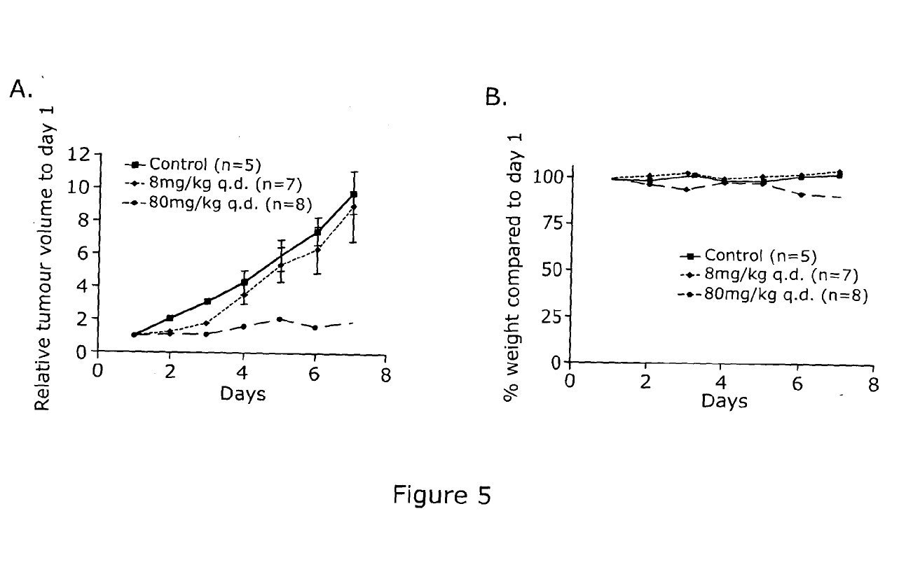

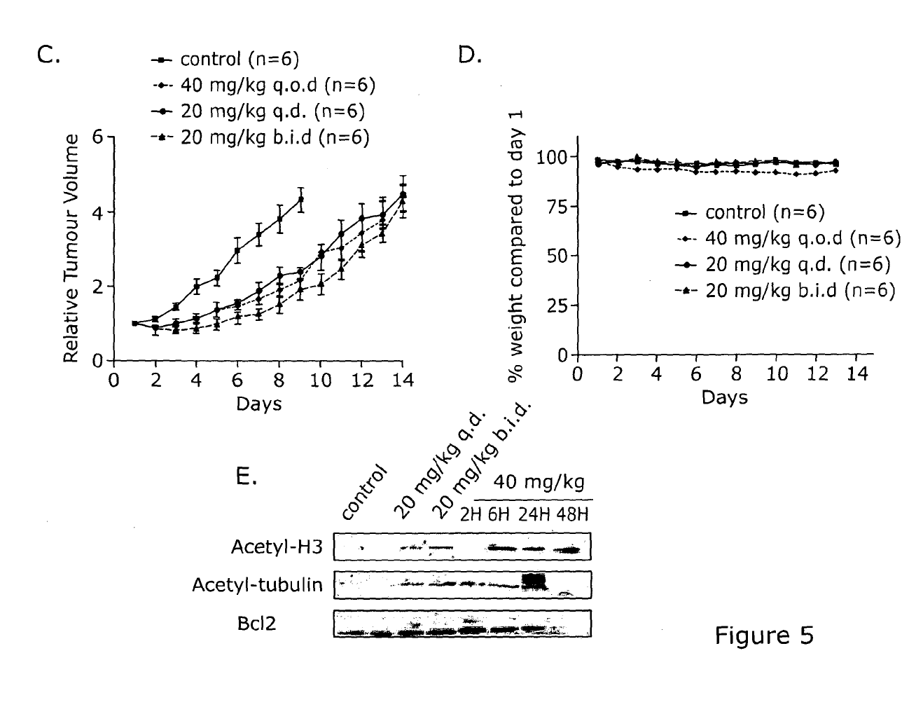

FIG. 5. Anti-tumour activity of HDAC-C1A in A2780 (A & B) and HCT116 (C & D) xenograft models (Tumours volumes relative to tumour volume at day 1 (A & C) and corresponding body weight (relative to body weight at day 1) (B & D)). E. Biomarkers screening at early time points following a single injection of HDAC-C1A in HCT116 xenograft at 40 mg/kg compared to a continuous 14 days treatment at the indicated dose. Tumours were excised 6 hours following the last injection.

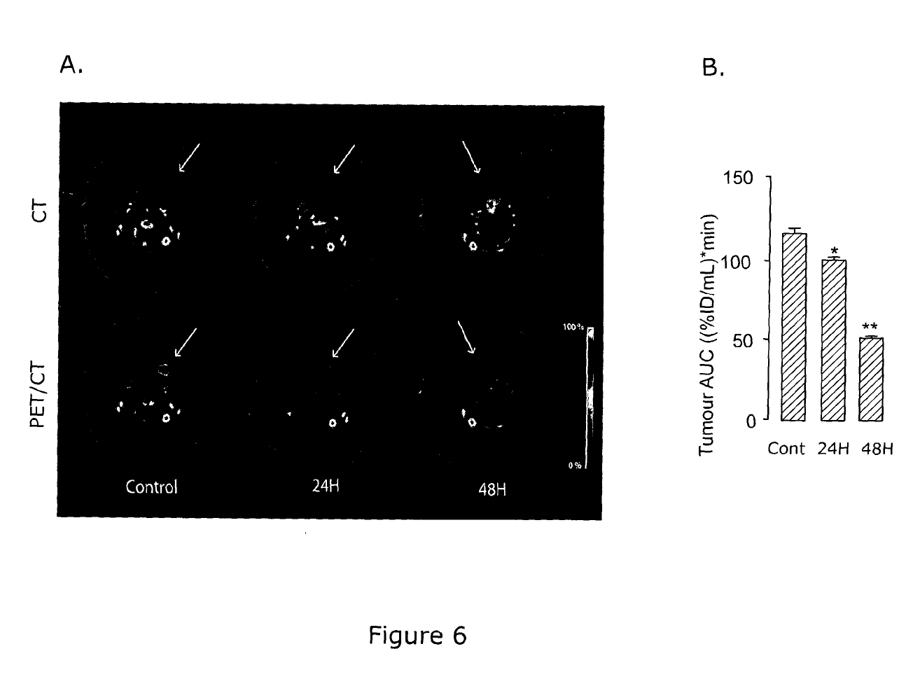

FIG. 6. [18F]-FLT uptake at 24 h and 48 h following a single injection I.P. of HDAC-C1A at 40 mg/kg in HCT116 xenograft model. A. PET/CT (bottom) and corresponding CT (top) images of the [18]-FLT uptake. For visualisation, cumulative images up to 60 minutes are displayed. The tumour is indicated by an arrow. B. Area under the curve data. The results are the mean+/−SEM of at least 4 animals per group.

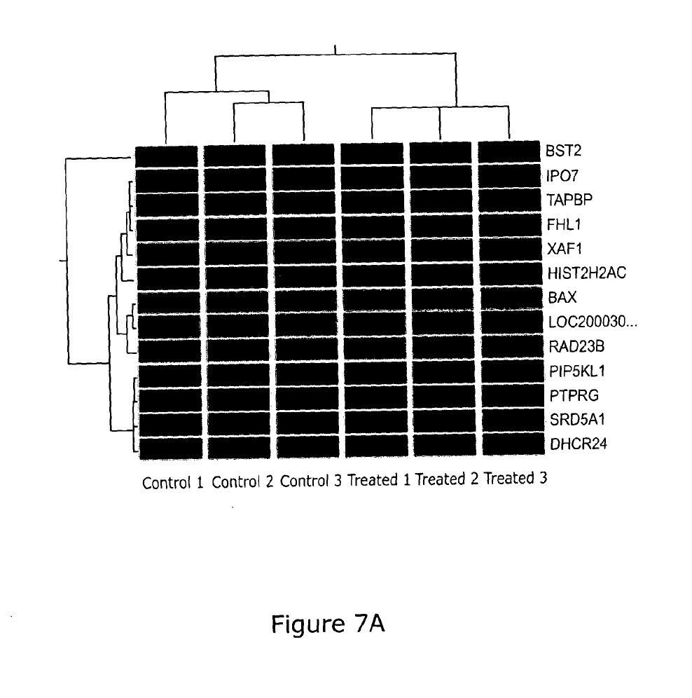

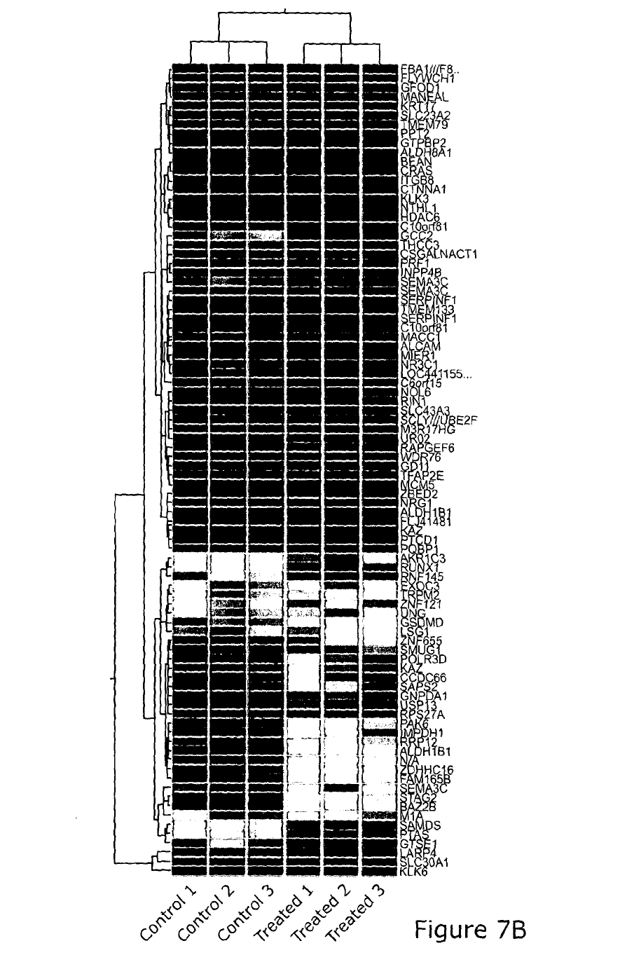

FIG. 7. Differential gene expression as expressed by mRNA levels following treatment with HDAC-C1A at 40 mg/kg using an affymetrix gene array. A. Differential gene expression after 24 hours following a single injection of HDAC-C1A at 40 mg/kg and compared with vehicle treated. B. Differential gene expression after 14 days of treatment at 40 mg/kg/day. The samples were excised after 6 hours following the last injection. A differential expression of 1.5 fold was chosen as a cut-off. Up and down-regulated genes are shown in grey-dark-black (depending on their level of de-regulation)

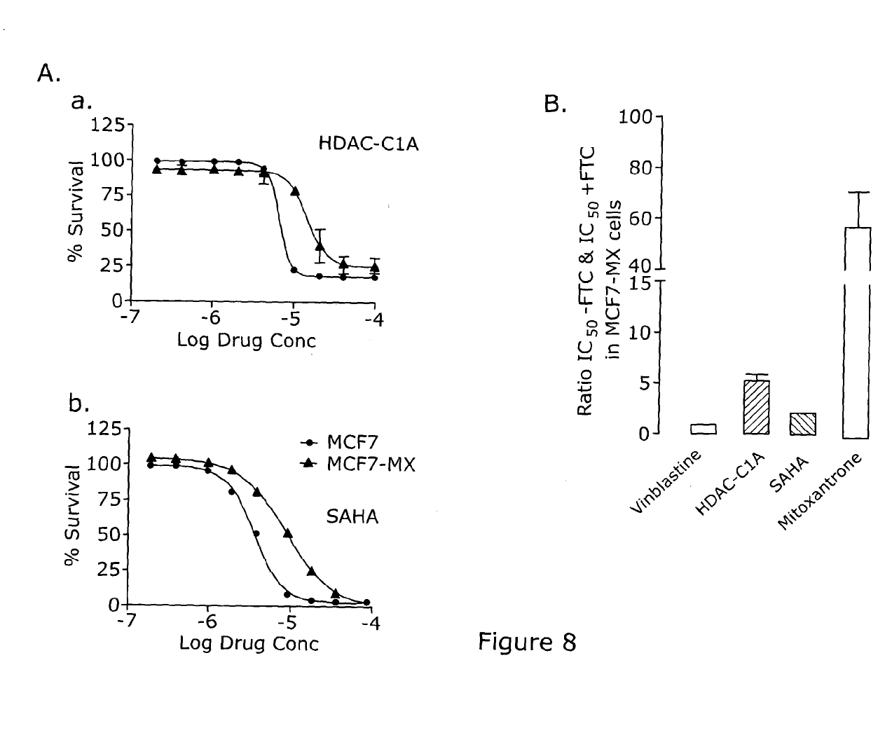

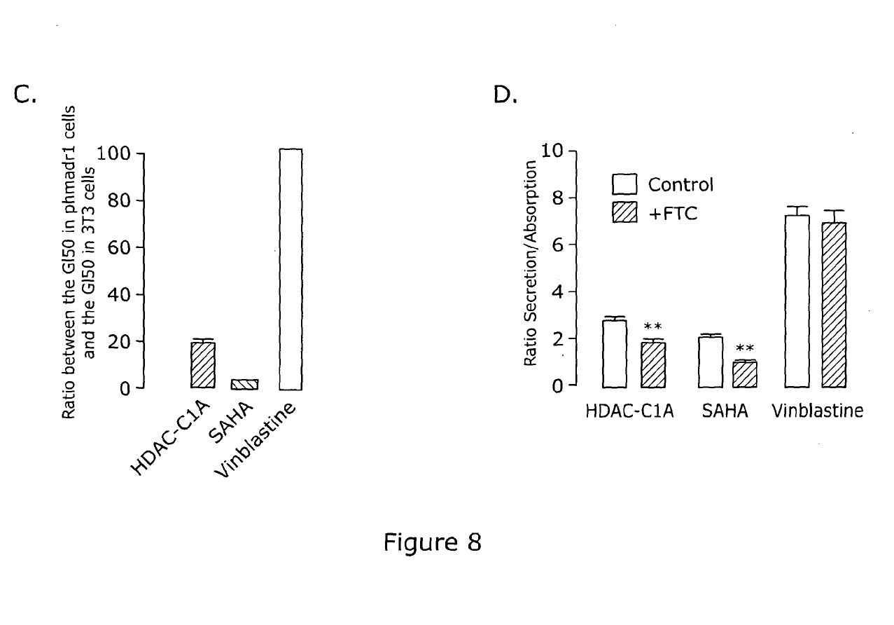

FIG. 8. Impact of the ABC transporters on the permeability and the cytotoxicity of HDAC-C1A and SAHA. A. Impact of ABCG2 on the cytotoxicity of HDAC-C1A and SAHA as determined by SRB assay in mitoxantrone resistant cell line, overexpressing ABCG2 (MCF7-MX) and in parental cell line MCF7. a. Growth inhibitory effect of HDAC-C1A. b. Growth inhibitory effect of SAHA. B. Impact of Fumitremorgin C (FTC), a specific inhibitor of ABCG2, on the cytotoxicity of the compounds in MCF7-MX cells. C. Impact of P-gp on the cytotoxicity of the different compounds in pHamdr1 cells, over-expressing the transporter and compared to its isogenic parental cell line 3T3. D. Impact of the ABC transporters on the permeability of HDAC-C1A and SAHA compared to the positive control vinblastine as determined by the transwell assay. A compound associated with a ratio between the secretion, i.e. permeability from the basal side to the apical side (Papp B-A) and the absorption, i.e. permeability from the apical to the basal side (Papp A-B) greater than 3, is considered as being actively efflux. FTC was used to reverse the active efflux, mediated by ABCG2 of both HDAC-C1A and SAHA (Vinblastine, is non substrate of ABCG2 and was used a negative control for FTC treatment).

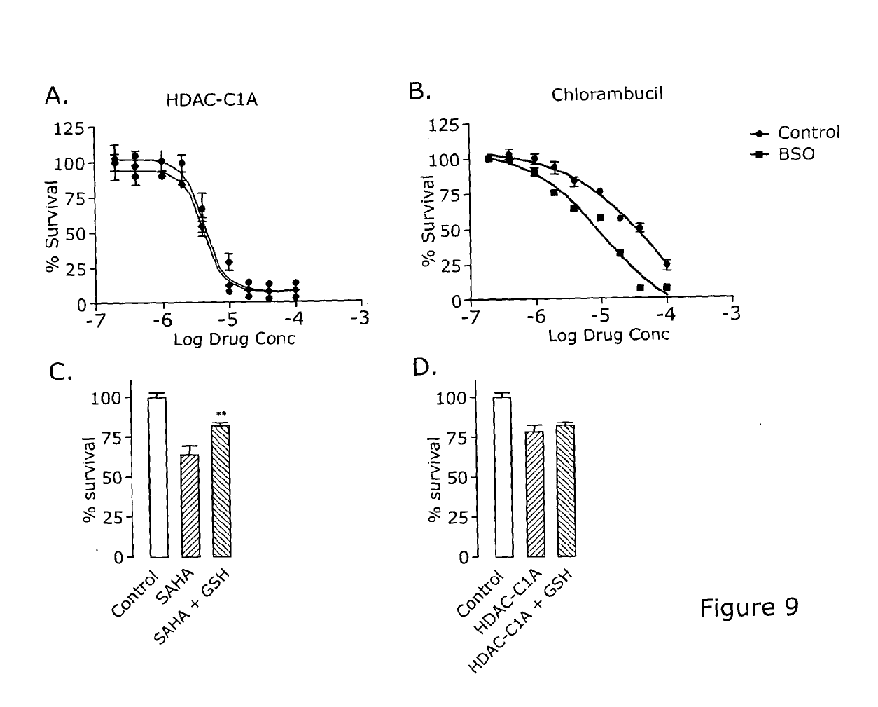

FIG. 9. HDAC-C1A is not detoxified by Glutathione in A549 and A2780 cells. A and B. Impact of BSO, an inhibitor of gamma-glutamylcysteine synthetase (gamma-GCS) that lowers intra-cellular glutathione levels on the growth inhibitory effect of HDAC-C1A (A) and the positive control chlorambucil (B) in A549 cells as determined by SRB assay. The cells were pre-treated or not with 10 μM of BSO for 24 hours. C and D. Impact of glutathione on the survival of A2780 cells following incubation with SAHA at 1 μM (C) and HDAC-C1A at 1 μM (D). The cells were co-incubated with 2.5 mM glutathione.

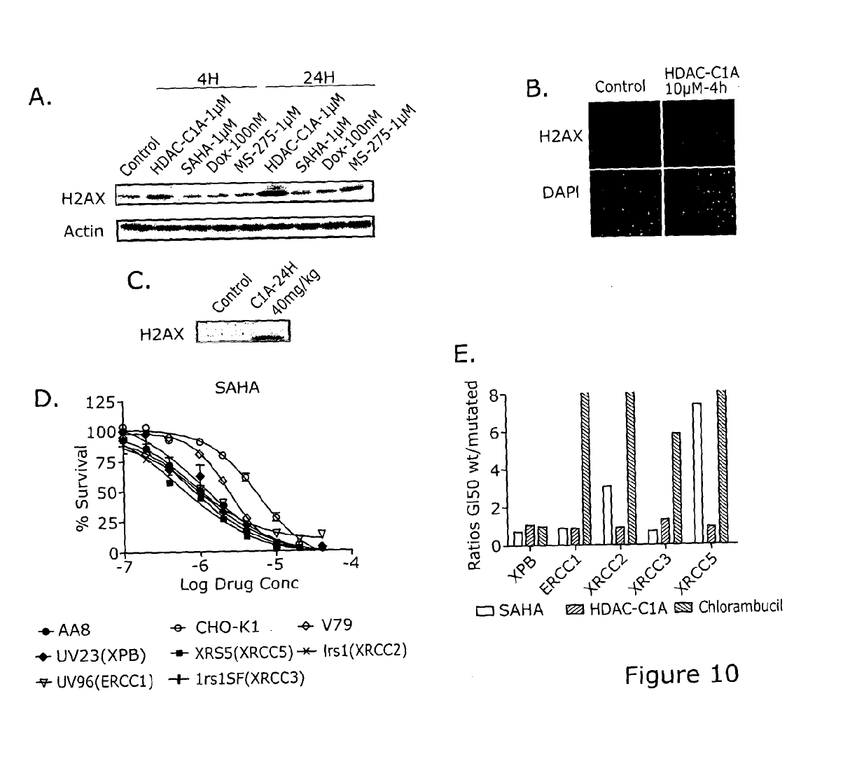

FIG. 10. Impact of the DNA repair machinery on the cytotoxicity of HDAC-C1A. A-C. HDAC-C1A treatment is associated with a γ-H2AX foci, marker of DNA damage. A. A2780 cells treated with different compounds at the GI50 at the indicated time. H2AX protein levels are revealed by western blotting. B. A2780 cells treated with HDAC-C1A at 10 μM for 4 h and stained for γ-H2AX foci by immunofluorescence. Bottom pictures shows the Dapi control, that stains the DNA in blue. C. Treatment with HDAC-C1A in vivo (HCT-116 xenograft model) at 40 mg/kg. H2AX proteins levels revealed by western blotting. D. Growth inhibitory effect of SAHA in cell lines deficient in the DNA repair machinery. UV23 and UV96 cell lines deficient in XPB and ERCC1, respectively involved in the nucleotide excision pathway. Irs1 and 1rs1SF deficient in XRCC2 and XRCC3, respectively involved in the homologous recombination repair pathway. XRS5 deficient in XRCC5 involved in the non homologous end joining pathway. Growth curves in parental cell lines AA8 (for UV23, UV96 and 1rs1SF), CHO-K1 (for XRS5) and V79 (for Irs1) are represented. E. Ratio between the GI₅₀ in the parental cell line and the GI₅₀ in the indicated deficient cell line. Chlorambucil, is represented as a positive control.

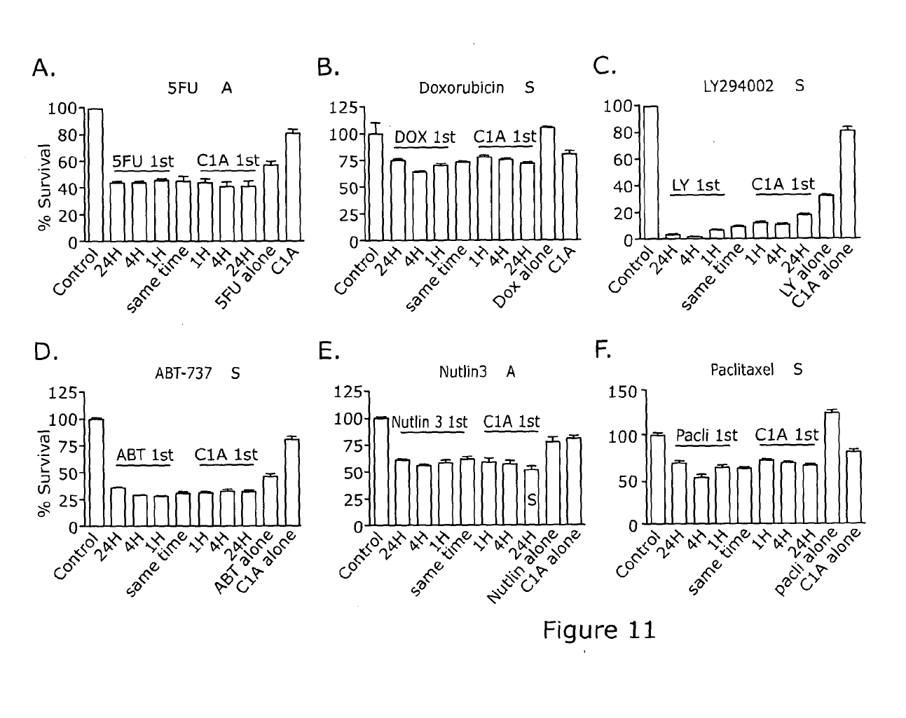

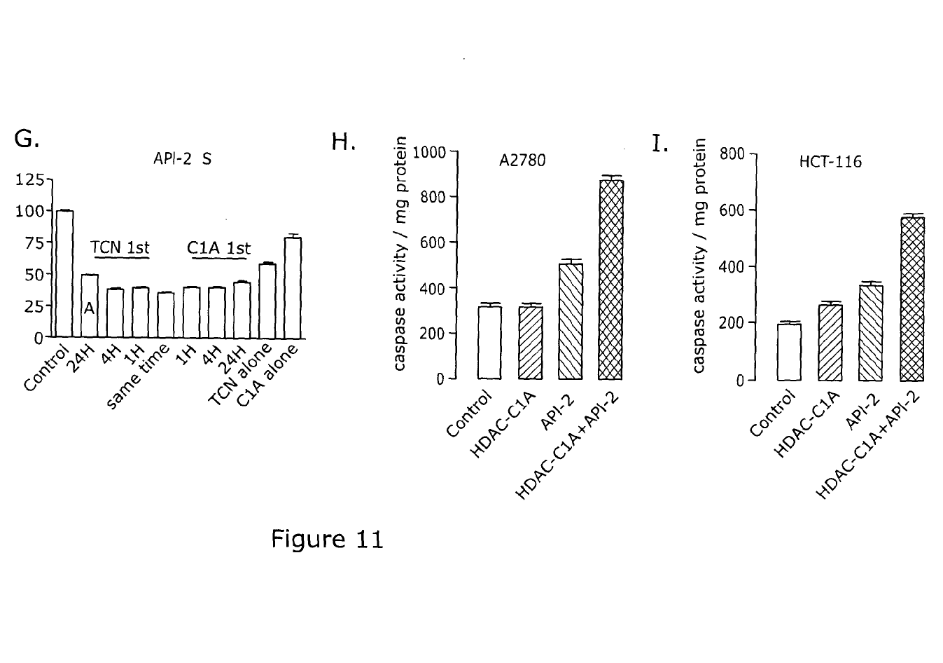

FIG. 11. Combination studies between HDAC-C1A and different class of compounds. A-G. Combination studies in HCT116 cells using the SRB assay. Compounds comprise of anti-metabolite (5-FU), DNA intercalating agent (doxorubicin), BCL2 inhibitor (ABT-737), inhibitor of mdm2 (nutlin-3), mitotic inhibitor (paclitaxel) and inhibitors of PI3K/AKT pathway (LY-29004 and API-2). “A” denotes an additive effect. “S” denotes a synergistic effect. Different treatment scheduling are shown. Synergism is demonstrated if the survival fraction following combination treatment is lower than (the survival fraction following HDAC-C1A treatment) times (the survival fraction following compound treatment). The results are mean of n=6. H and I. combination between HDAC-C1A and API-2 in A2780 cells (H) and in HCT116 cells (I) as determined by caspase 3/7 activity assay. The concentrations of HDAC-C1A were chosen at the GI50 for each cell line (0.7 μM and 3.7 μM for HDAC-C1A in A2780 and HCT116 cells, respectively and 2.2 μM and 4.4 μM for API-2 in A2780 and HCT116, respectively).

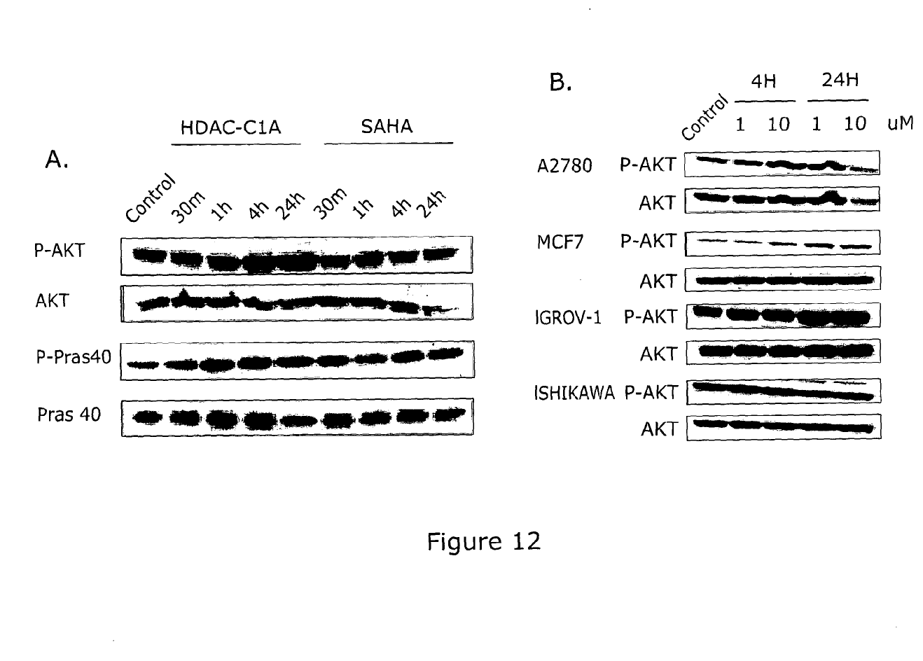

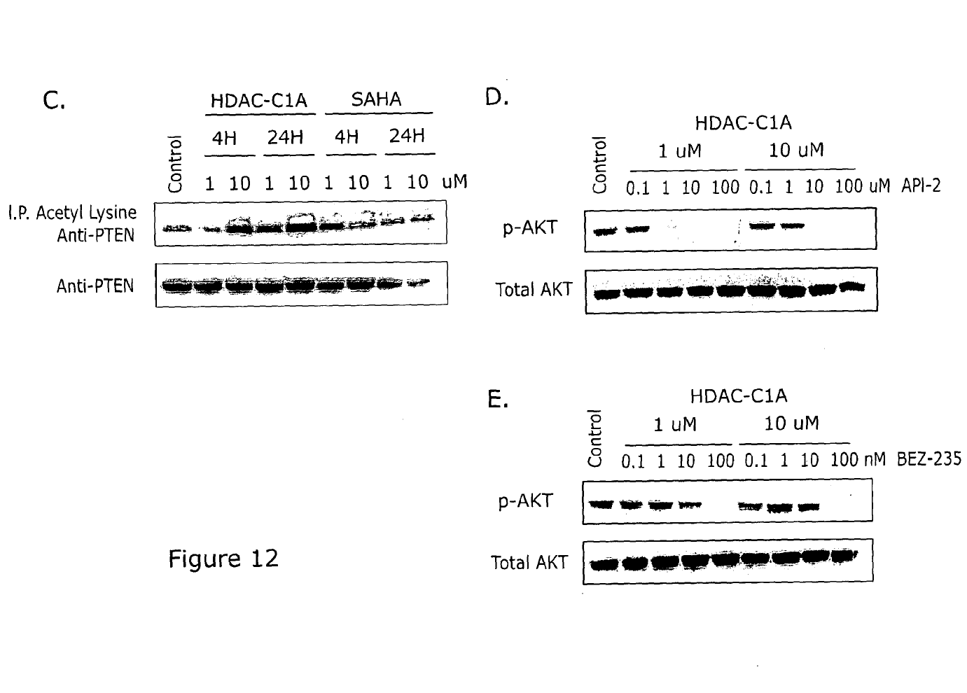

FIG. 12. HDAC-C1A treatment is associated with an increase of phopsho-AKT that can be reversed by PI3K/AKT/mTOR inhibitors. A. Time dependent increase of phospho-AKT following treatment with HDAC-C1A at 10 μM in contrast with SAHA at the same concentration in HCT116 cells. Similarly, Phospho-Pras 40 seems to increase following treatment with HDAC-C1A. Densitometry data could reinforce this statement (ratio between the total form and the Phospho form of Pras40 that decreases at 24 hours). B. Time and dose dependent increase of Phosho-AKT in a panel of cancer cell lines. No difference could be seen in endometrial cancer cell line, ISHIKAWA. C. Time and dose dependent increase of the acetylated form of PTEN following treatment with HDAC-C1A in HCT-116 cells in contrast with SAHA. D. API-2 can partially reverse the increase of Phospho-AKT mediated by HDAC-C1A in HCT-116 cells. API-2 and HDAC-C1A were co-incubated at the indicated time and concentrations. E. BEZ-235 can fully reverse the increase of Phospho-AKT mediated by HDAC-C1A in HCT-116 cells. BEZ-235 and HDAC-C1A were co-incubated at the indicated time and concentrations.

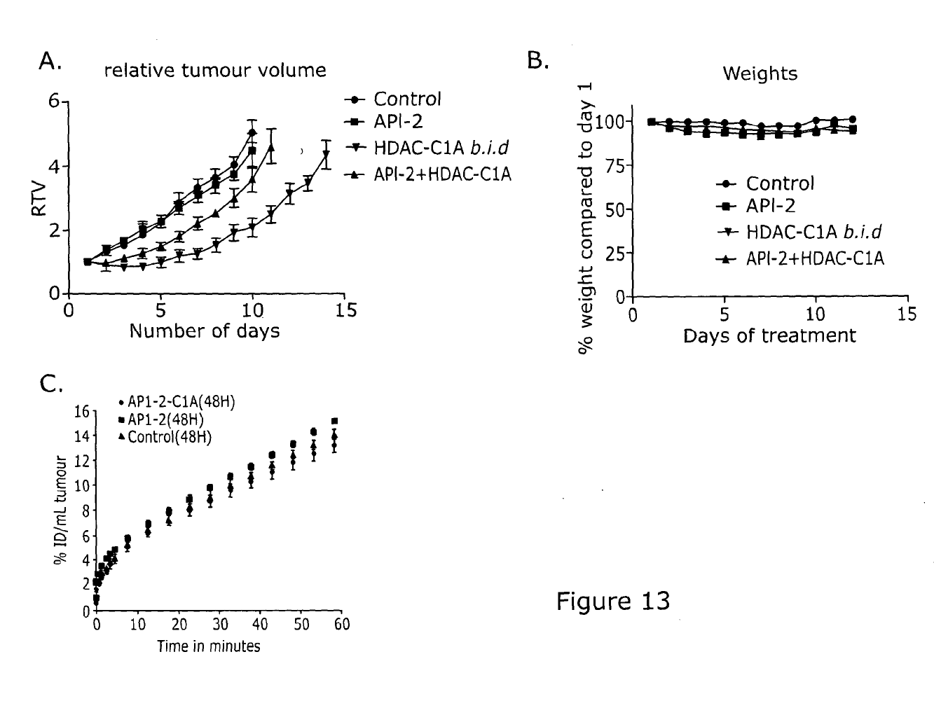

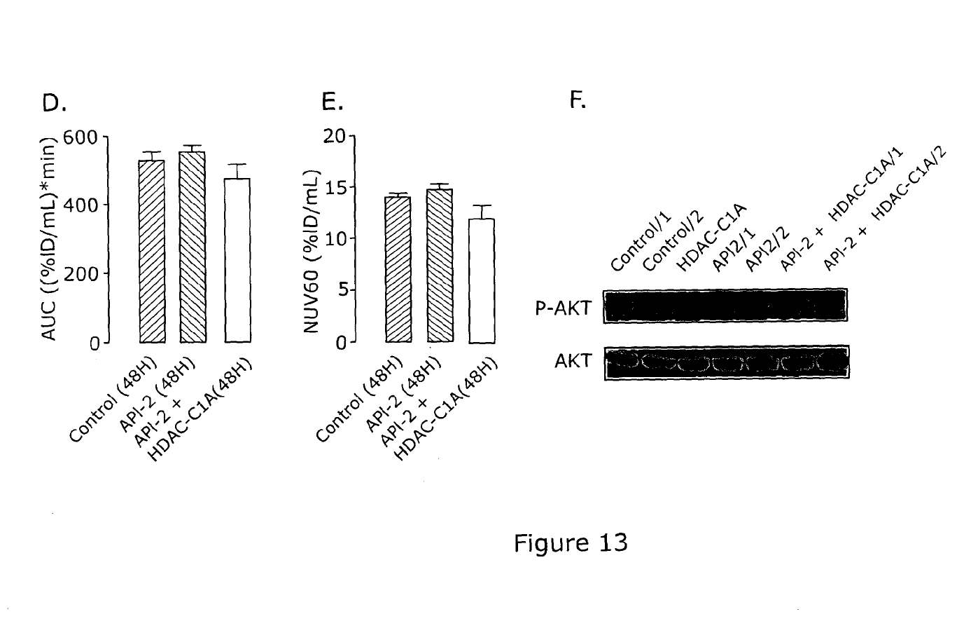

FIG. 13. Effect of HDAC-C1A in combination with API-2 on anti-tumour activity and 18[F]FLT-PET tumour uptake. A and B: Anti-tumour activity of HDAC-C1A in combination with API-2 for 14 days. A. relative tumour volume to day 1. HDAC-C1A was given I.P. at 20 mg/kg b.i.d. API-2 was given I.P. at 1 mg/kg q.d. B. Corresponding body weights throughout the course of the experiment. C-E. [18F]FLT-PET in HCT-116 tumour bearing mice following 48 H treatment with the combination HDAC-C1A (40 mg/kg) and API-2 (1 mg/kg). C. Time activity curve represent [18F]FLT uptake over 60 minutes. Results are mean of at least 3 animals. D. Area under the curve, [18F]FLT uptake in the tumour over 60 minutes. E. NUV60, [18F]FLT uptake in the tumour at 60 minutes. F. Phospho-AKT levels in HCT-116 tumours, 24 hours after a single injection of HDAC-C1A at 40 mg/kg or API-2 at 1 mg/kg or in a combination (HDAC-C1A—40 mg/kg+API-2—1 mg/kg). The levels of total AKT are represented as loading control.

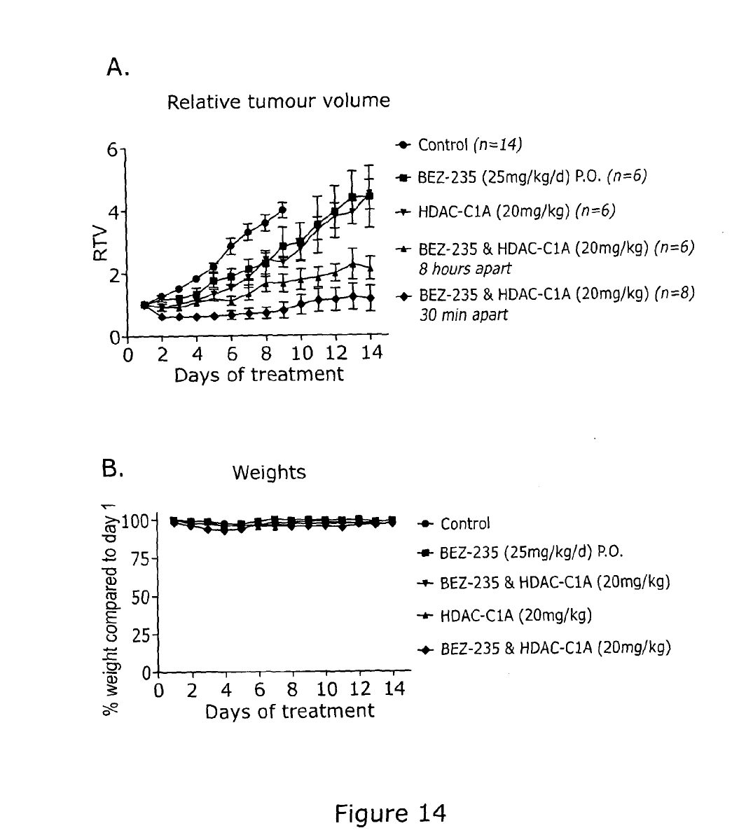

FIG. 14. Anti-tumour activity of HDAC-C1A in combination with BEZ-235 for 14 days. A. relative tumour volume to day 1. HDAC-C1A was given I.P. at 20 mg/kg b.i.d. BEZ-235 was given P.O. at 25 mg/kg q.d. BEZ-235 was given first in the morning, The second dose of HDAC-C1A was given I.P. 8 hours later or 30 min later. B. Corresponding body weights throughout the course of the experiment.

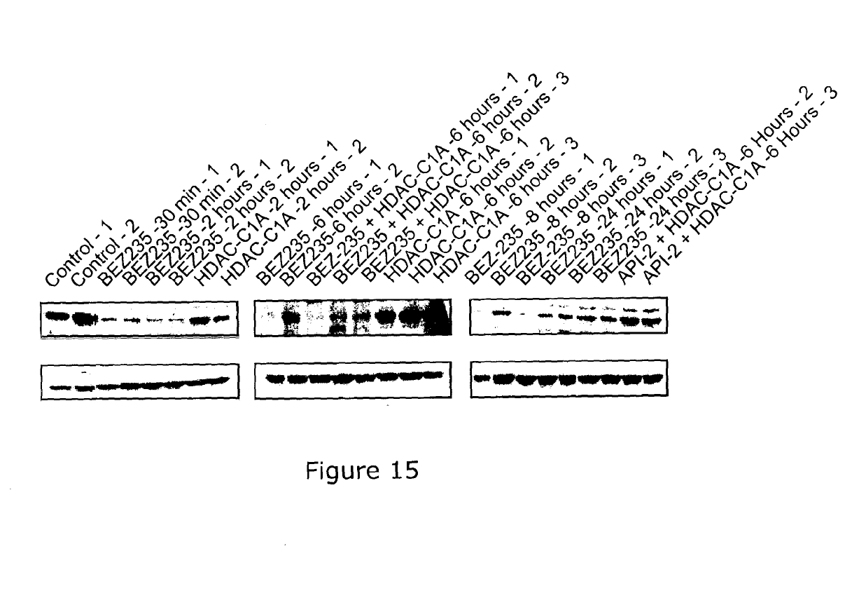

FIG. 15. Phopho-AKT levels following a single injection of BEZ-235 (25 mg/kg) P.O. or HDAC-C1A (20 mg/kg) I.P. or in combination (BEZ-235+HDAC-C1A). The levels of Phospho AKT following a combination regimen with API2 at 1 mg/kg and HDAC-C1A at 20 mg/kg are also represented.

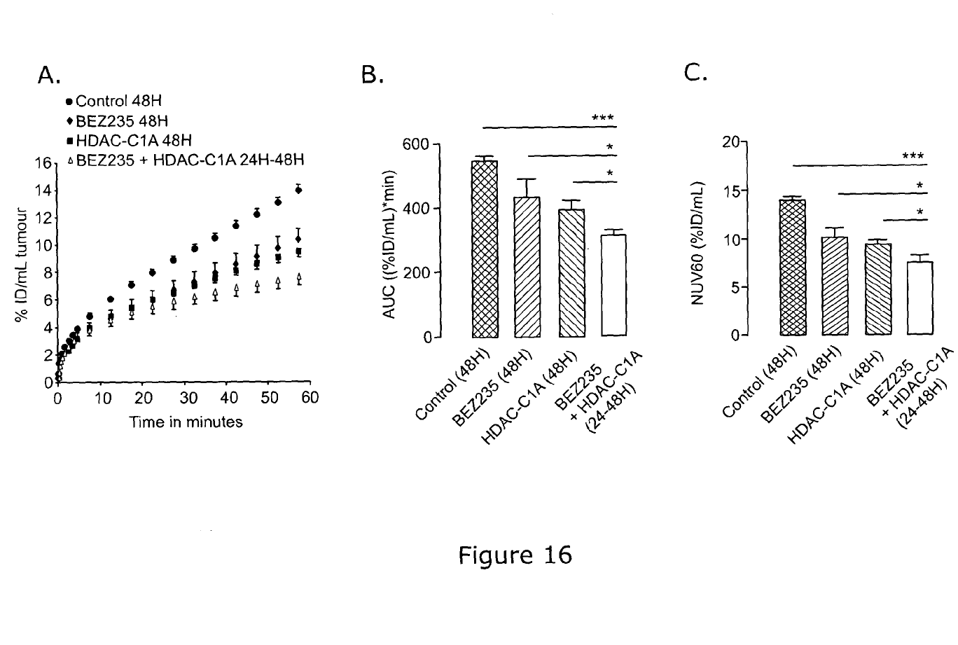

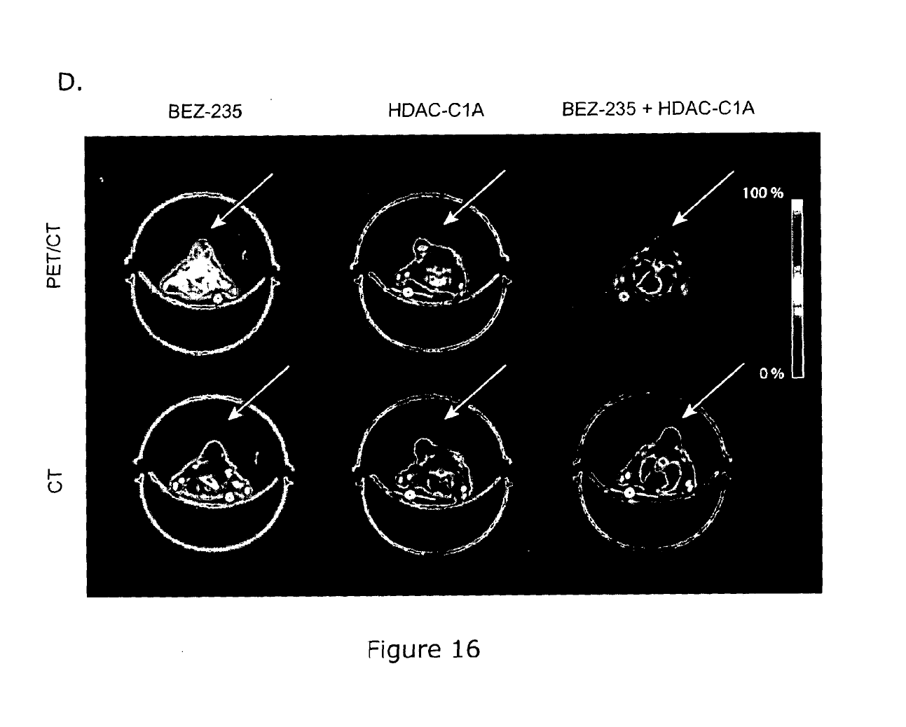

FIG. 16. [18F]FLT uptake following treatment with BEZ235 and in combination with HDAC-C1A after 48 hours in a HCT116 xenograft model. Mice were treated with either BEZ235 alone at 20 mg/kg/day for 2 days P.O., with 1HDACC1A at 20 mg/kg/day for 2 days I.P. or with a combination HDAC-C1A (20 mg/kg/day for 1 or 2 days)+BEZ-235 (20 mg/kg/day for 1 or 2 days). BEZ235 was given first and 30 min later, mice received an injection of HDAC-C1A. A. time activity curve represent [18F]FLT uptake over 60 minutes. Results are mean of at least 3 animals. B. Area under the curve, [18F]FLT uptake in the tumour over 60 minutes. C. NUV60, [18F]FLT uptake in the tumour at 60 minutes. D. Cross section (axial plane) of mice representative of different treatment cohorts. The top pictures show [18F]FLT uptake pictures (PET/CT), the bottom represent the corresponding CT scan. The arrows point the tumour.

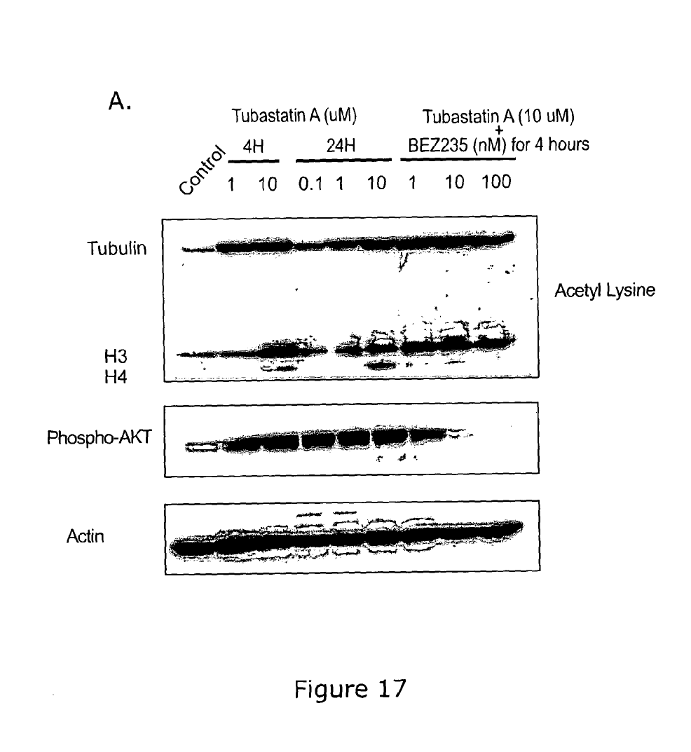

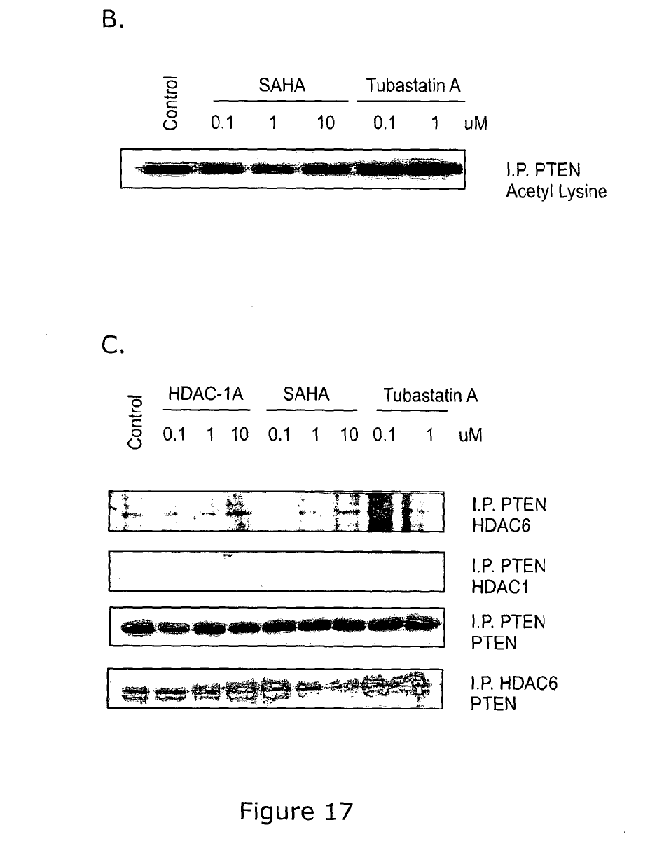

FIG. 17. A. Dose and time impact of Tubastatin A on acetylated form of tubulin and histones H3 and H4 and on phospho-AKT levels that could be reversed with BEZ-235 in HCT-116 cells. B. Dose dependent impact of tubastatin A on the acetylated form of PTEN in contrast with SAHA in HCT116 cells after 24 hours of incubation at the indicated dose C. HCT116 were immunoprecipitation with PTEN and stained with HDAC1 or HDAC6 (lines 1-3), immunoprecipitation with HDAC6 and stained with PTEN (line 4). No HDAC1 could have been detected in contrast with HDAC6. There was no effect of the different inhibitors on the different protein levels.

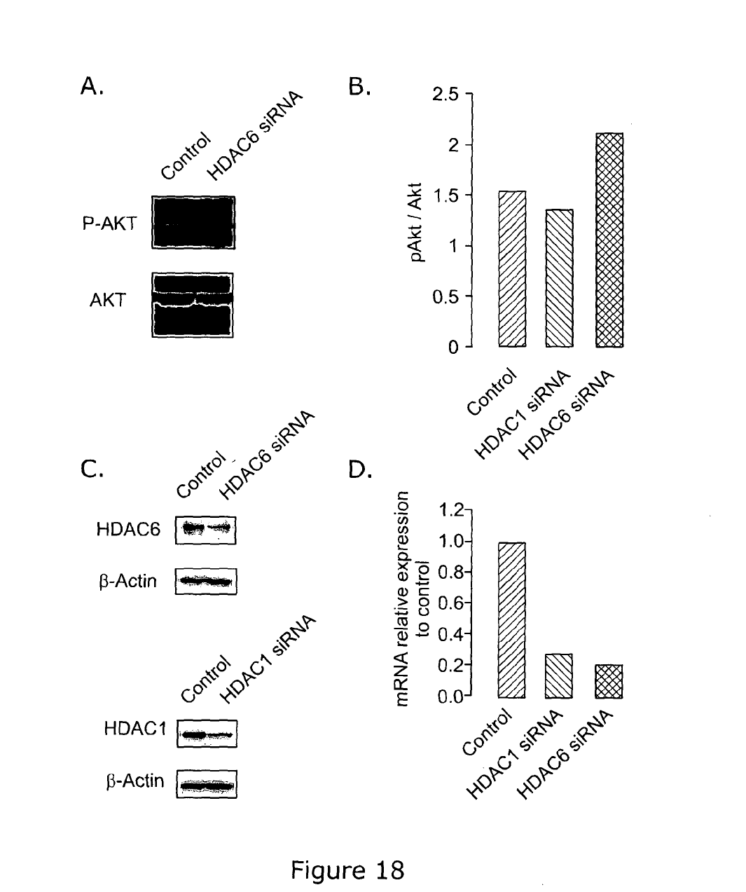

FIG. 18. HDAC6 silencing by SiRNA is associated with an increase of Phospho-AKT in HCT 116 cells. Cells were incubated with 50 nM siRNA for 48 hours (both HDAC1 and HDAC6). (A) Level of Phospho-AKT following incubation with HDAC6 SiRNA as determined by western blotting. (B) Ratios of Phospho-AKT over total AKT following incubation with HDAC6 siRNA and HDAC1 siRNA. (C). HDAC1 and HDAC6 protein expression following incubation with siRNA. (D) HDAC1 and HDAC6 mRNA levels following incubation with siRNA as determined by qPCR

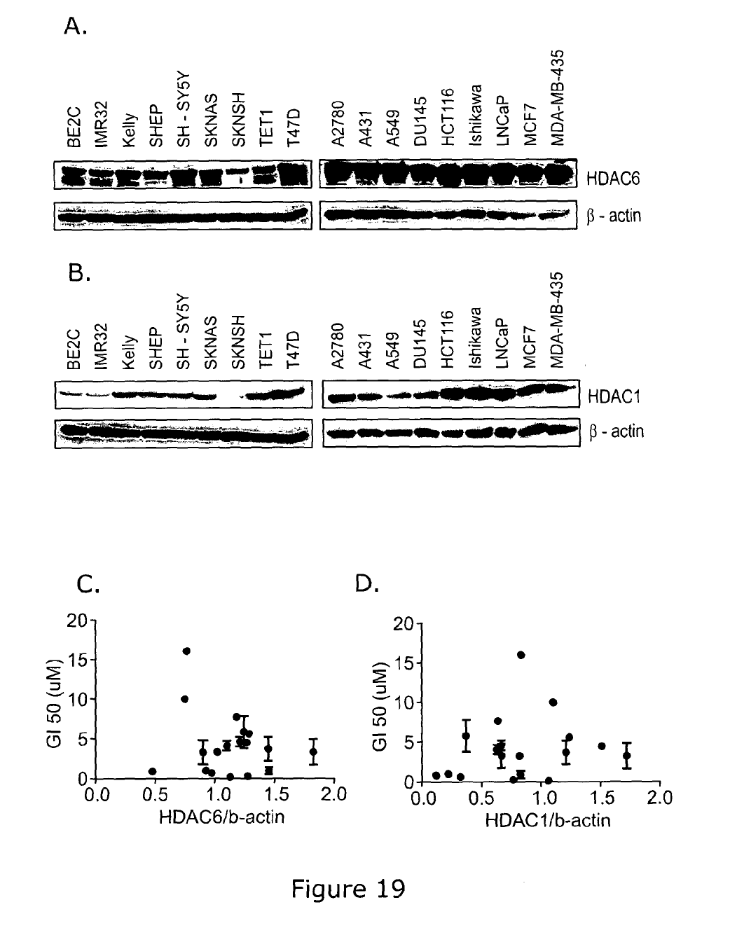

FIG. 19. HDAC6 and HDAC1 protein expression in a panel of cancer cell lines as determined by western blotting. A. HDAC6 protein expression. B. HDAC1 protein expression. C and D. Relationship between HDAC6 (C), HDAC1 (D) and the growth inhibitory effect (GI50) of HDAC-C1A.

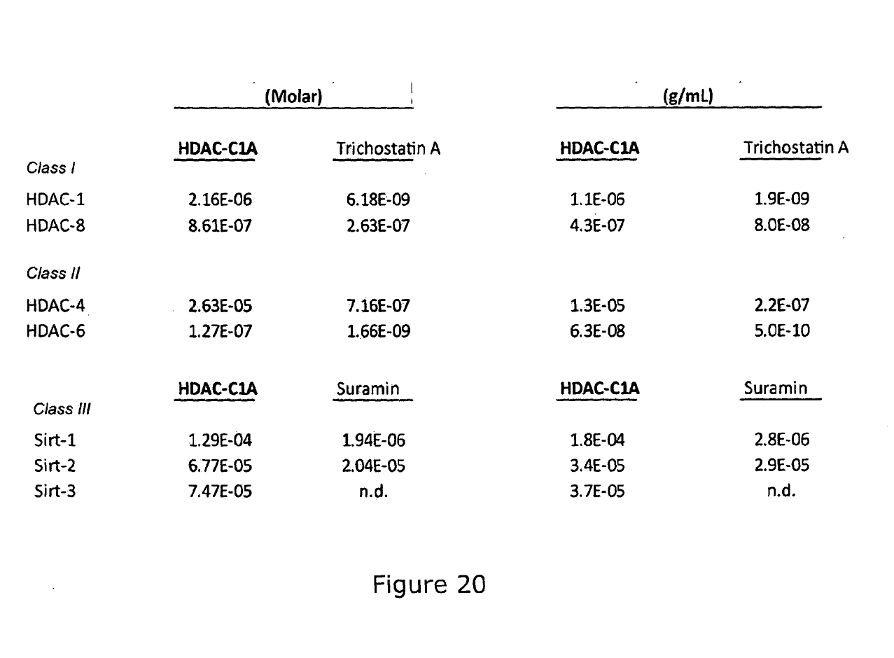

FIG. 20 (Table 1): Enzyme inhibition activity using HeLa nuclear extract and the Fluor-de-Lys kit.

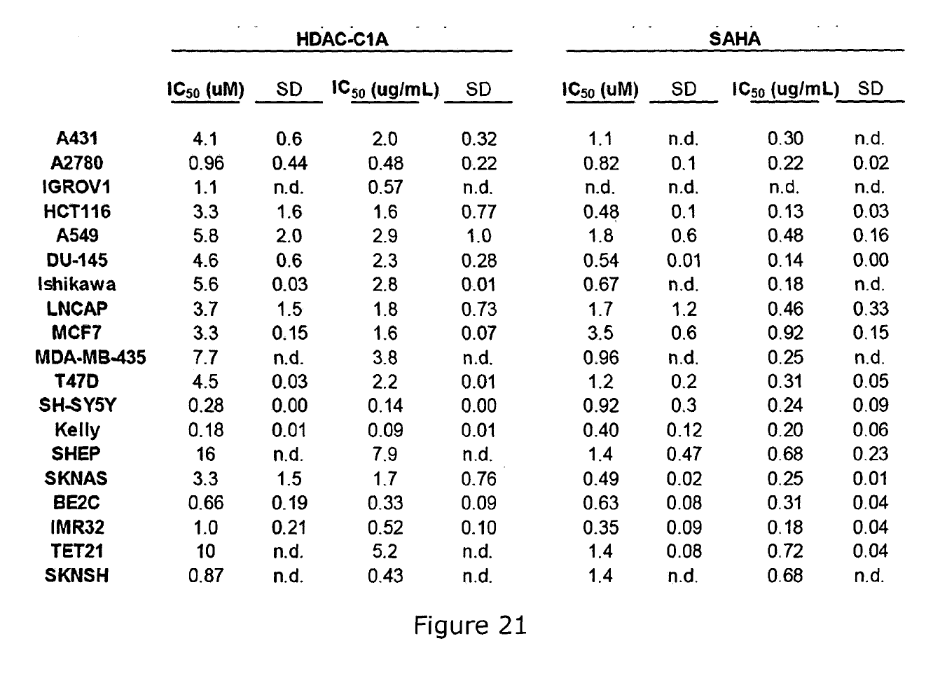

FIG. 21 (Table 2): Growth inhibitory effect of HDAC-C1A and SAHA in a panel of cancer cell lines following 72 hours incubation as determined by the SRB assay.

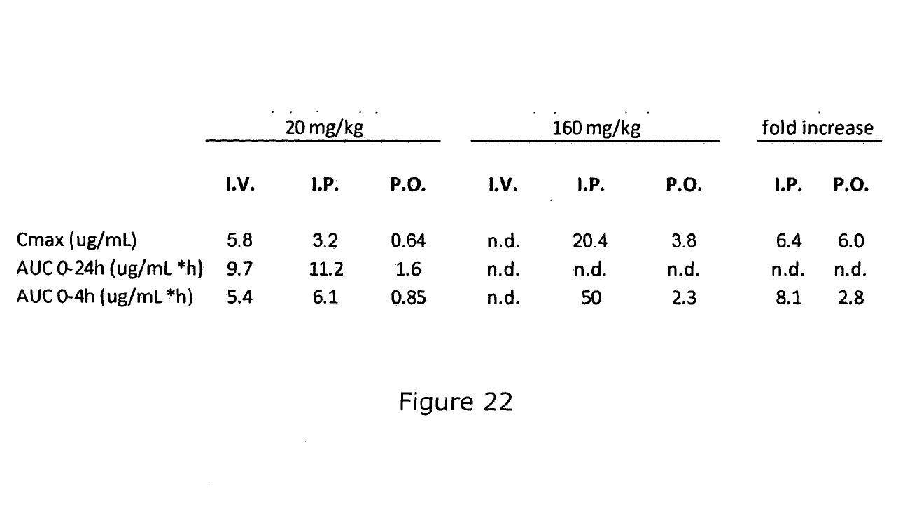

FIG. 22 (Table 3): Pharmacokinetic data of HDAC-C1A following a single injection.

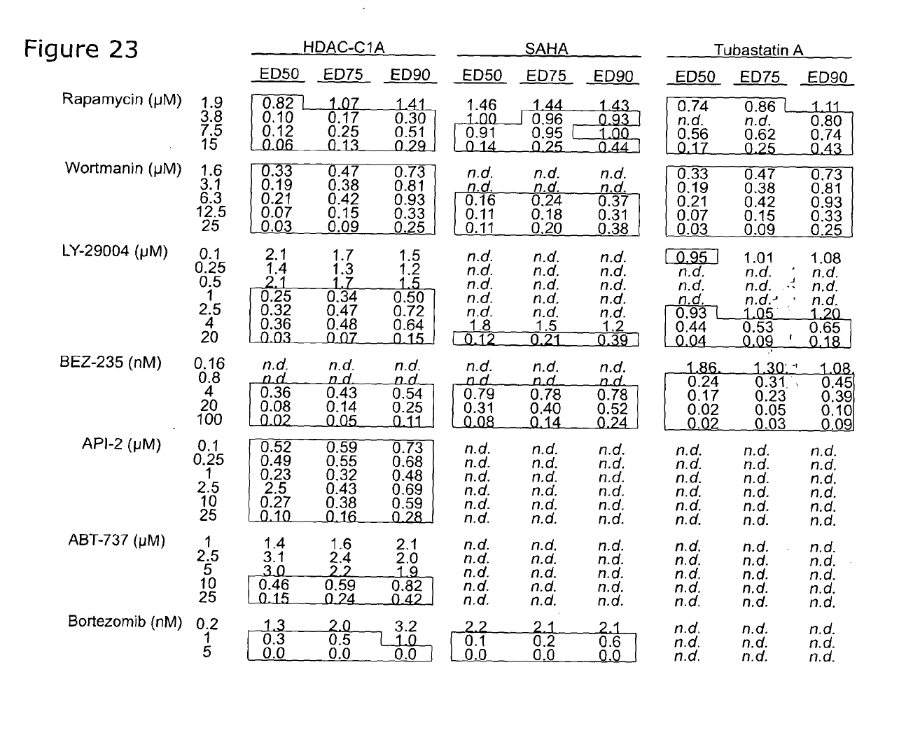

FIG. 23 (Table 4). Combination index (CI) between HDAC-C1A, SAHA and Tubastatin A and the different inhibitors of the PI31K/AKT/mTOR signalling pathway (Rapamycin, wortmanin, LY-29004, BEZ-235, API-2), BCL2 inhibitor (ABT-737) and the proteasome inhibitor (Bortezomib) in HCT116 following 72 hours Incubation. The different CI have been determined using the effect dose at 50%, 75% and 95% using the SRB assay. Results are mean of n=3. CI<1 demonstrates synergism (highlighted in gray). CI=1, additive effect and CI>1, an antagonistic effect. The CI has been calculated using CalcuSyn.

### EXAMPLE 1

### HDAC-C1A, a Novel HDAC6 Irreversible Inhibitor in Combination with Inhibitors of PI3K/AKT/mTOR Signalling

Histone deacetylase (HDAC) enzymes exert control over gene transcription and cell cycle progression and their inhibition has recently emerged as an efficacious strategy to treat cancer. However, current HDAC inhibitors have been linked to a shared undesirable toxicological profile.

### HDAC-C1A has High Affinity Towards HDAC6.

HDAC-C1A inhibited class I, II and sirtuins, with highest affinity for HDAC6 (IC₅₀=63 ng/mL) (Table 1), an HDAC subtype thought to be associated with low toxicity; HDAC6 knockout does not lead to embryonic lethality. The drug irreversibly inhibited HDAC from HeLa cell extract; in HCT116 cells inhibition of enzyme activity as assessed by levels of acetyl-histone H3, H4 and acetyl-tubulin was maintained after washout demonstrating an irreversible mechanism (FIG. 1). HDAC-C1A treatment was associated with a dose and time dependent increase of histone (histone H3 and H4) and non-histone targets α-tubulin-client protein of HDAC6) that was maintained at 4 hours after washout; acetylation was lost by 4 h with clinically licensed HDAC inhibitor SAHA (FIG. 1). HDAC-C1A was associated with a dose and time dependent increase of acetyl form of HSP90 (another client protein of HDAC6) when tested at 4 h and 24 h and at 1 μM and 10 μM in HCT116 cells. HSP90 protein expression was not altered (FIG. 2).

### HDAC-C1A has Marked In Vitro and In Vivo Anti-Tumour Activity.

HDAC-C1A inhibited the growth of a panel of 19 cancer cell lines with a mean GI₅₀ of 2±0.4 μg/mL (Table 2). The drug was stable after oral, parenteral and intravenous administration for 24 h (last time point examined). Plasma concentrations 2 orders of magnitude above the GI₅₀ were obtained. The Cma and AUC₀₋₂₄ₕ following i.p. injection (20 mg/kg) were 3.2 μg/mL and 11.2 μg/mL*h, respectively. When given orally, the Cmax and AUC₀₋₂₄ₕ were 0.7 μg/mL and 94 μg/mL*h, respectively. This route of administration could be privileged in the treatment of sensitive tumour types such as neurobastoma (SH-SY5Y and KELLY), that are associated with GI₅₀ of 0.09 μg/mL and 0.14 μg/mL, respectively. The oral bioavailability of HDAC-C1A was 16%. The stability of HDAC-C1A was evaluated across a range of acidic pH, that would mimic the pH of the stomach (down to pH 2) and, cell free studies, demonstrated a relative acidic degradation (FIG. 3). The acid instability of HDAC-C1A could therefore be improved by enteric coating as a means to avoid breakdown in the stomach. The pharmacokinetic of HDAC-C1A was dose proportionate following i.p. injection (160 mg/kg); was dose proportionate with Cmaxand AUC₀₋₄ₕ (Table 3). Regarding efficacy in the rapidly growing A2780 human ovarian cancer xenograft model, HDAC-C1A treatment was associated with a Tumour Growth Delay (TGD₂ₓ) of 3±0.3 days and a Tumour Growth Inhibition (TGI) of 74% compared with vehicle when given i.p. at 80 mg/kg/day (FIG. 5.A). In some animals 10% body weight loss was observed at 7 days and treatment was stopped (FIG. 5.B). HDAC-C1A was also tested in HCT116 human colon cancer xenograft model using a refined dose and scheduling. It was associated with TGD₂ₓ of 5.7±1.4 days and a TGI of 78% compared with vehicle when given i.p. at 20 mg/kg b.i.d. (FIG. 5.C). This dose was non-toxic (no reduction in body weight) (FIG. 5.D). The acetylated form of α-tubulin (biomarker of response to HDAC6 inhibition) could be observed after 14 days of treatment and could be detected as early as 6 hours after the 1^{st} injection (FIG. 5.E.). The acetylated form of Histone 3 (biomarker of response of class I histones) could be observed as early as 2 hours after the 1^{st} injection and remain after 14 days of treatment (FIG. 5.E). The anti-apoptotic factor Bcl2 was down-regulated after 48 hours (FIG. 5.E).

### [18F]-FLT Could be Used to Predict Response to Chemotherapy as Early as 48 h.

We also assessed the potential of [¹⁸F]fluorothymidine positron emission tomography ([¹⁸F]FLT-PET) to measure early response to HDAC-C1A treatment in HCT116 xenograft bearing mice (FIG. 6). There was a 2-fold decrease in tumour [¹⁸F]FLT uptake in animals treated continuously for 48 h compared to vehicle treated controls; the area under the normalized [¹⁸F]FLT time versus activity curve was 117±6.9 before treatment and decreased to 106±5.5 (P=0.02) at 24 hours and 54±5 (P=0.0001) at 48 hours after initiating treatment (FIG. 6.B).

### Minor Effect on the Regulation of the Genes.

The impact of HDAC-C1A treatment on gene expression was evaluated in vivo in a HCT116 xenograft model using the affymetrix human genome array plate. Of the 20 000 genes tested, only 20 genes were deregulated after 24 hours of treatment with HDAC-C1A at 40 mg/kg (17 including pro-apototic factors like BAX and XAF1 were up-regulated) (FIG. 7.A). After 14 days of treatment at 40 mg/kg every other day, 132 genes (<0.7%) were deregulated by at least 1.5 fold when compared to the control group, treated with vehicle (FIG. 7.B). RAD23B, an inhibitor of the proteasomal activity, was up-regulated by ˜1.8 fold; this factor was recently shown to be a predictive bio-marker of response to HDAC inhibitors such as SAHA, PXD101 and dipsepetide (the greater the expression, the greater the response) but is also involved in the DNA repair pathway and was shown to be up-regulated in colon cancer cell lines, resistant to 5-FU. Other resistance-related genes such as ALDH1 and UNG were also upregulated (by ˜1.6 fold and ˜1.7 fold, respectively); ALDH1 is a specific bio-marker of cancer stem cell population and UNG is involved in the base excision repair pathway.

Interestingly, few genes involved in proliferation such as KLK3, encoding for prostate specific antigen, were down regulated (by ˜1.5 fold). Pan inhibitors SAHA and LBH589 were also shown to down-regulate this particular gene but failed to do so in patients due to poor PK. Genes involved in cell invasion and metastasis (KLK6, ERAS, MACC1, VCAN), were also down regulated (between −1.5 and ˜1.7 fold). Other genes involved in apoptosis (Gasdermin D) or differentiation (K AZ) were up regulated (both by ˜1.5 fold). Similarly, SYNDECAN-2, previously shown to be associated with reduced anchorage independent growth following treatment with trichostatin A, was down regulated (by ˜1.7 fold). Interestingly some genes involved in proliferation and invasion, such as KRT17, NRG1, CTNNA1, CXCL14, SERPINA1, FLMN2, FAT4, were de-regulated (between −1.5 and ˜1.7 fold).

### HDAC-C1A is a Weak Substrate of the ABC Transporters.

The impact of ABC transporters on the cytotoxicity of HDAC-C1A was assessed using cell lines with differential expression of the ABC transporters and by using inhibitors. With regards to ABCG2, MCF7-MX—the mitoxantrone resistant cell lines (overexpressing ABCG2)—were 2.8- and 2.5-fold more resistant than parental cell line MCF7 to HDAC-C1A and SAHA, respectively (FIG. 8.A). Incubation with FTC, specific inhibitor of ABCG2 in MCF7-MX cells reversed the resistance to HDAC-C1A and SAHA by 5.2- and 3.9-fold, respectively. Both compounds can be considered as relatively poor substrates of ABCG2 with regards to the positive control mitoxantrone with GI₅₀ ratios of 120 between the 2 cell lines and a reversal of resistance of 70-fold with FTC (FIG. 8.B). With regards to P-gp, 3T3 fibroblasts transfected with cDNA over-expressing P-gp were 17- and 2.4-fold more resistant to HDAC-C1A and SAHA, respectively than the parental cell line (3T3, transfected with cDNA containing an empty vector). Similarly, both compounds can be seen as weak substrates of P-gp with regards to the positive control vinblastine with GI₅₀ ratios between the 2 cell lines >100 (FIG. 8.C). Both HDAC-C1A and SAHA were found to be non-substrates of MRP1 as the pre-treatment with MK-571 did not have any effect on their cytotoxicity in A549 cells.

The impact of the ABC transporters on permeability was further assessed using a caco-2 transwell assay. A compound is considered to be actively effluxed by the ABC transporters when the ratio between the permeability from the basal side to the apical side (Papp_{B-A}) and the permeability from the apical to the basal (Papp_{A-B}) side is greater than 3. HDAC-C1A and SAHA were associated with Papp_{B-A}/Papp_{A-B} ratios of 2.8 and 2.1, respectively (FIG. 8.D). The pre-treatment with FTC reduced the ratios to 1.8 and 1.1 for HDAC-C1A and SAHA, respectively. Finally, the expression of the ABC transporters was evaluated on both proteins and mRNA levels in HCT116 xenograft models after 14 days of treatment with HDAC-C1A and compared with vehicle treated group. No difference could be demonstrated.

HDAC-C1A was associated with an average permeability (Papp_{A-B}) across the caco-2 cells of 5.1×10⁻⁶ cm/s (±0.2) that could predict a relatively good bioavailability similar to SAHA (Papp_{A-B}=14±0.2×10⁻⁶ cm/s) shown to be orally bioavailable.

### HDAC-C1A is not Detoxified by GSH.

The impact of BSO, an inhibitor of the synthesis of GSH was evaluated in A549 cells. No difference was seen in contrast with the positive control chlorambucil (GI₅₀ ratios of 8.5) (FIG. 9). GSH, at 2.5 mM was demonstrated to have an antagonistic effect on the cytotoxicity of SAHA but not HDAC-C1A in A2780 cells.

### The DNA Repair Machinery is not Involved in the Resistance of HDAC-C1A.

The chemical structure of HDAC-C1A contains a nitrogen mustard that is similar to chlorambucil and melphalan, shown to interact with DNA. HDAC-C1A treatment is associated with a time and a dose dependent increase of γH2AX foci formation both in vitro and in vivo (FIG. 10.A-C). Few genes involved in DNA repair machinery (RAD23B and UNG) were up-regulated in the tumours with reduced responsiveness to HDAC-C1A after 14 days of treatment (FIG. 7). We wanted to test whether HDAC-C1A, could interact with DNA (inducing DNA damage and subsequent toxicity) and if cell lines deficient in the DNA repair machinery would be more sensitive to HDAC-C1A. CHO cells deficient in XPB and ERCC1 of the nucleotide excision repair pathway, cells deficient in XRCC2 and XRCC3 of the homologous recombination repair pathway (HRR) and cells deficient in XRCC5 of the non homologous end joining repair pathway were found to be as sensitive as the parental proficient cell lines (FIG. 10.D-E). In contrast, cells deficient in XRCC2 and XRCC5 were 3.1- and 7.4-fold more sensitive to SAHA when compared to proficient cells. Chlorambucil was found to be associated with GI₅₀ ratios >100 between cells proficient and deficient in ERCC1, XRCC2 and XRCC5 and a GI₅₀ ratio of 7.8 in cells deficient in XRCC3 (FIG. 10.E). Also, the level of mRNA and protein expression of RAD51 (involved in HRR pathway) did not increase following treatment with HDAC-C1A in vitro nor in vivo.

In Vitro, HDAC-C1A Synergizes with Inhibitors of PI3K/AKT/mTOR Signalling

Synergistic effect has been demonstrated using AKT inhibitors API-2, BEZ-235 LY294002, wortmanin, rapamycin, the BCL2 inhibitor ABT-737, and the proteasome inhibitor bortezomib (Table 4). The combination of those compounds was compared to the pan inhibitor SAHA. Overall, SAHA synergises as well with the inhibitors of the PI3K/AKT/mTOR signalling pathway. However, the synergism between HDAC-C1A and the different inhibitors was demonstrated at lower concentrations than with SAHA. For instance, there is antagonistic effect between SAHA and rapamycin (concentrations below 7.5 μM) at the effective dose 50 (ED₅₀) whereas HDAC-C1A synergises as low as 1.9 μM. Similarly, SAHA synergises with LY-294002 (sometimes written as LY-29004) at concentrations greater than 4 μM whereas only 1 μM of LY-294002 is needed to synergise with HDAC-C1A. Tubastatin A, an HDAC6 specific inhibitor synergises as well with inhibitors of the PI3K/AKT/mTOR pathway. The synergy was demonstrated at even lower concentrations (0.1 μM of LY-294002 is sufficient to synergise with Tubastatin A). The schedule of administration was also investigated and a pre-incubation with PI3K/AKT/mTOR inhibitors 4 hours before incubation with HDAC-C1A proved optimal for growth inhibition (FIG. 11.C). We were also able to demonstrate that PI3K/AKT/mTOR inhibitors synergise not only the growth inhibitory effect but also on the apoptotic effect of HDAC-C1A in A2780 and HCT 116 cells (FIG. 11.H and 11.1). In contrast, only an additive effect was demonstrated with P53 inducer Nutlin-3 and the anti-metabolite drug 5FU (FIG. 11).

In Vitro, HDAC-C1A is Associated with an Increase of P-AKT in Colon Cancer Cell Lines (HCT116) that can be Reversed by AKT Inhibitors API-2 and BEZ-235.

Treatment with HDAC-C1A increased P-AKT (i.e. phosphorylated Akt) by 1.5-fold at 10 μM at 4 h (starting at 30 min) and 2-fold increase at 24 h in contrast SAHA was shown to have no effect on P-AKT in this specific cell line (FIG. 12.A). (P-AKT levels were also increased in ovarian cancer cell lines IGROV-1 and A2780, and breast cancer cell line MCF7, but not in endometrial cancer cell line, Ishikawa) (FIG. 12.B). This particular cell line has been shown to express high levels of PTEN mRNA but did not express PTEN protein because of protein truncations (i.e., PTEN-null) (X Wan, et al, (2002) Cell death and differentiation, 9: 414-420). HDAC-C1A was associated with a dose and time dependent increase of acetyl form of PTEN in vitro when tested at 4 h and 24 h and at 1 μM and 10 μM in HCT116 cells; in contrast SAHA had no effect (FIG. 12.C). PTEN protein expression was not altered. In vivo, on the gene array, the mRNA levels of PTEN were not altered in HCT116 xenograft model following treatment with HDAC-C1A after 1 and 14 days when compared with vehicle treated group (FIG. 7).

Both API-2 and BEZ-235 treatment were associated with a dose dependent decrease of P-AKT levels in HCT116 cells. After 4 hours treatment, API-2 inhibits P-AKT with an IC₅₀ of 1 μM and BEZ-235 at 10 nM (FIGS. 12.D and 12.E.). API-2 and BEZ-235 both decreased P-AKT levels when combined with HDAC-C1A. When combined with 10 μM HDAC-C1A the IC₅₀ for API-2 was 10 μM and 50 nM for BEZ-235. FIG. 12 shows that a higher dose of HDAC-C1A can decrease the effect of API-2 on reducing HDAC-C1A-induced phosphorylation of AKT whereas 1 and 10 μM HDAC-C1A and were similarly modulated by BEZ-235 (much lower dose of this compound used compared to API-2).

API-2 inhibits AKT by binding to its pleckstrin homology domain and blocks AKT membrane translocation (Kim et al, (2010) J. Biol. Chem., 285(11): 8383-8394). It is possible that an activating feedback loop from the mTOR pathway could have an impact on P-AKT levels that could not reverse completely the increase of P-AKT induced by HDAC-C1A when API-2 is used.

In contrast, BEZ-235 is a dual inhibitor of the PI3K and the mTOR pathway (Maira et al, (2008) Mol. Cancer. Ther. 7 (7): 1851-1863) and acts to reduce HDAC-C1A mediated p-AKT increase at a much lower concentration than API-2. Therefore, the more general mode of action of BEZ-235 may negate any potential feedback loop. In any event, BEZ-235 is able to prevent more efficiently the phosphorylation of AKT induced by HDAC-C1A than API-2 in vitro. This may simply be a result of its lower IC₅₀ (i.e. increased activity/affinity) for HDAC-C1A-induced p-AKT reduction.

Thus, it is thought that the action of any potential feedback mechanism in cells will be sufficiently diminished by effective reduction in p-AKT mediated by the “AKT inhibitor”.

In Vivo, API-2 is Neither Associated with Decreases of P-AKT Nor Increased Anti-Proliferative in Combination with HDAC-C1A.

When combined with API-2 at 1 mg/kg/d, HDAC-C1A treatment at 20 mg/kg bid was associated with a Tumour Growth Delay (TGD₂ₓ) of 2.7±1.3 days and a Tumour Growth Inhibition (TGI) of 54% compared with vehicle in a HCT116 xenograft model (FIG. 13.A). Treatment with API-2 alone did not have any effect on tumour growth. [¹⁸F]FLT-PET in tumour bearing mice following 48 H treatment with the combination HDAC-C1A and API-2 was unremarkable in comparison to control animals (FIG. 13.C-E.). Tumours from combined HDAC-C1A and API-2 treated mice still showed increased P-AKT similar to HDAC-C1A-only treated animals and P-AKT levels in API-2 only treated tumours were no different from controls (FIG. 13.F). These data indicate that at the schedule and dose used, API-2 was incapable of reducing the effect of HDAC-C1A on P-AKT. Moreover, we could argue that P-AKT levels were higher in vivo following co-treatment of API-2 and HDAC-C1A that suggests signs of antagonism in vivo with API-2.

In vivo, the unfavourable pharmacokinetics of API-2 may not allow it to achieve the minimum desirable concentration (10 μM) at the tumour site. Therefore, it is likely that API-2 had a low potency due to properties unrelated to its affinity for AKT. Finally, API-2 is effective only in tumours that over-express AKT, therefore potentially having low in vivo affinity for AKT (Yang et al (2004) Cancer Res. 64: 4394-9).

Nevertheless, in vitro API-2 acted to reduce p-AKT induced by HDAC-C1A only at relatively high concentrations, particularly in comparison with BEZ-235. Therefore, the low in vivo activity of API-2 may simply be a result of its low affinity. Also, API-2 treatment is associated with many side effects that could be linked to API-2 being not absolutely specific for AKT inhibition. Thus, the low in vivo activity of API-2 may have several explanations.

In Vivo, BEZ235 is Associated with a Decrease of P-AKT Levels that Synergise with HDAC-C1A.

HDAC-C1A treatment at 20 mg/kg/day was associated with a TGD₂ₓ of 3.8±1.3 days and a TGI of 69% (FIG. 14). When combined with BEZ-235 at 20 mg/kg/day (given 8 hours apart), the TGD₂ₓ was 8.2±1.3 days and a TGI of 74%. When the same combination was given 30 minutes apart, the treatment was associated with a TGI of 115% TGD₂ₓ could not be calculated. BEZ-235 alone at 20 mg/kg/d was associated with a TGD₂ₓ of 3.4±1.9 days and a TGI of 21%. These data indicated that, when combined appropriately, drugs that inhibit P-AKT can positivity modulate the activity of HDAC-C1A.

Phospho-AKT levels were screened in HCT116 tumours following a single injection of BEZ235 over time and compared to HDAC-C1A alone and in combination. There was a rapid down-regulation of P-AKT by 30 minutes following a single injection of BEZ235 at 25 mg/kg (FIG. 15). Persistent inhibition was still observed 2 hours after treatment, with a partial recovery to basal levels from 6 hours post treatment. The single treatment with HDAC-C1A at 20 mg/kg shows an up-regulation of P-AKT remarkable at 6 hours. No P-AKT was detected using the combination regimen at 6 hours. This demonstrates that the inhibition of BEZ-235 inhibits efficiently P-AKT in vivo in contrast with API-2 (cf-supra) and that a specific scheduling may aid in achieving the maximum desirable effect.

### The Efficacy of the Combination Regimen can be Monitored as Early as 24H-48H Using [¹⁸F]FLT-PET.

The efficacy of the combination HDAC-C1A (20 mg/kg/day) and BEZ235 (25 mg/kg/day) could be monitored with [¹⁸F]FLT-PET in HCT116 tumour bearing mice as early as 24H-48H. There was a 1.7-fold decrease in tumour [¹⁸F]FLT uptake in animals treated continuously for 24 h-48 h compared to vehicle treated controls; the area under the normalized [¹⁸F]FLT time versus activity curve was 542±17 before treatment and decreased to 312±22 (P=0.0002) at 24-48 hours after initiating treatment (FIG. 16.B).

HDAC6 siRNA Increases Levels of Phosphorylated AKT

FIG. 18 shows that P-AKT is increased in HCT116 cells after HDAC6 siRNA treatment for 48 hours.

### HDAC6 Protein Expression is not a Predictive Biomarker for HDAC-C1A Sensitivity

HDAC6 protein expression is required for efficient tumorigenesis (Lee et al. (2008) Cancer Res. 68; 7561) but, little is known about HDAC6 expression, being a predictive biomarker for anti-tumor activity. HDAC6 protein expression was evaluated in a panel of 18 cancer cell lines but did not correlate with the growth inhibitory effect of HDAC-C1A (FIG. 19). Similarly, HDAC1 protein expression did not show any correlation with HDAC-C1A sensitivity.

### The Mechanism of Action of HDAC-C1A, is a Common Feature of HDAC6 Inhibitors.

We have shown that HDAC-C1A is associated with increased acetylation of α-tubulin, acetyl HSP90 and acetyl PTEN with only a minor effect on the regulation of the genes (less than 0.7% of the genes have been de-regulated after 14 days of treatment in vivo). We believe that the acetylation of PTEN, via the inhibition of HDAC6, leads to an increase of P-AKT. We have shown that the combination treatment with PI3K/AKT/mTOR results in a synergistic effect on both growth inhibition and apoptosis in vitro and in vivo. Tubastatin A has recently been described as the most potent and specific inhibitor of HDAC6 (Butler et al, (2010) JACS 132: 10842-10846). It is also associated with a time dependent increase of P-AKT that could be reversed by the addition of BEZ235 (IC₅₀=10 nM) (FIG. 17.A). No effect of BEZ235 could be seen on the acetylated status of α-tubulin and histones H3 and H4. Tubastatin A was also associated with a dose dependent increase of the acetylated form of PTEN (FIG. 17.B). Finally, synergism was demonstrated with all the PI3K/AKT/mTOR inhibitors at low concentrations (Table 4).

The co-localisation of PTEN with HDAC6 (in contrast with HDAC1) has been shown for the first time by immunoprecipitation and emphasises the proposed mechanism, depicted in the diagram below (FIG. 17.C).

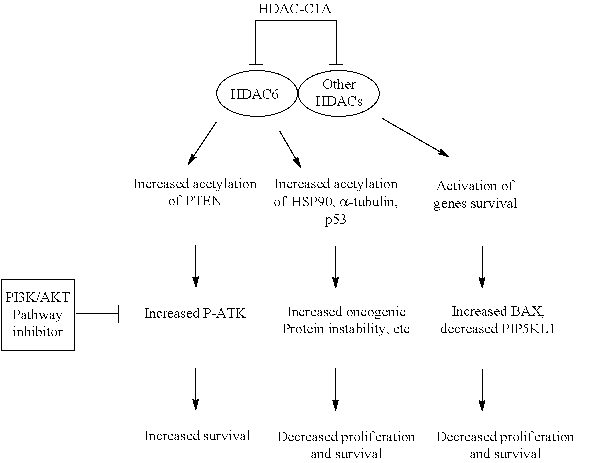

Diagram showing the proposed strategy to overcome the resistance (i.e non responsiveness) mediated by HDAC inhibitors by the use of AKT inhibitors (i.e, BEZ-235) leading to a total tumour growth inhibition in a colon cancer HCT116 cell line. The inhibition of HDAC6, (e.g. by HDAC-C1A) is associated with an increase of the acetylated form of α-tubulin and HSP-90 that subsequently lead to increased expression of pro-apoptotic proteins (i.e BAX) and reduced expression of anti-apoptotic proteins (PIP5KL1), This cascade of events prevents proliferation and induces apoptosis of tumours cells. However, in parallel, the inhibition of HDAC6 is associated with an increase of the acetylated form of PTEN, that consequently leads to an increase of P-AKT, involved in the survival and subsequent resistance. We propose that PI3K/AKT pathway inhibitors and pro-apoptotic drugs can overcome the resistance induced by the HDAC6 inhibitors.

1. A pharmaceutical composition comprising a Histone Deacetylase 6 (HDAC6) inhibitor and an AKT inhibitor with a pharmaceutically acceptable excipient, adjuvant, diluent or carrier.

2-3. (canceled)

4. The pharmaceutical composition of claim 1, further comprising one or more anti-cancer agents.

5. The pharmaceutical composition of claim 4, wherein the anti-cancer agents are selected from the group comprising apoptosis inducing drugs, chemotherapy agents, biologic therapies, proteasome inhibitors, and HSP90 inhibitors.

6-7. (canceled)

8. A method of preventing or treating a neuro-degenerative condition or an autoimmune disease in a patient needing such preventing or treating, comprising administering to the patient an effective amount of the pharmaceutical composition of claim 1.

9. A method of preventing or treating cancer, a neuro-degenerative condition, or an autoimmune disease, or a combination thereof, in a patient needing such preventing or treating and who has been administered an AKT inhibitor or a Histone Deacetylase 6 (HDAC6) inhibitor, comprising administering an effective amount of a HDAC inhibitor to the patient in need thereof who has been administered an AKT inhibitor, or an effective amount of an AKT inhibitor to the patient in need thereof who has been administered a Histone Deacetylase 6 (HDAC6) inhibitor.

10-13. (canceled)

14. A method of preventing or treating cancer, a neuro-degenerative condition, or an autoimmune disease, or a combination thereof, in a patient needing such preventing or treating, comprising the step of administering concurrently or sequentially a Histone Deacetylase 6 (HDAC6) inhibitor and an AKT inhibitor to the patient in need thereof.

15. (canceled)

16. The method of claim 14 for treating cancer, wherein the Histone Deacetylase 6 (HDAC6) inhibitor and the AKT inhibitor are administered concurrently or sequentially with one or more anti-cancer agent.

17. The method of claim 16, wherein the cancer is breast cancer, ovarian cancer, prostate cancer, bowel cancer, lung cancer, neuroblastoma, leukaemia, lymphoma or melanoma.

18-19. (canceled)

20. The pharmaceutical composition of claim 1, wherein the HDAC6 inhibitor is a compound of formula I, or a compound of formula IX;
wherein formula I is 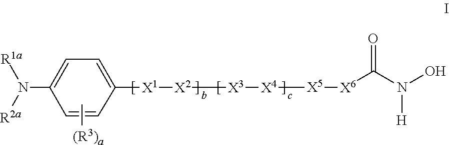
wherein
R^{1a} represents C₁₋₄ alkyl (which latter group is optionally substituted by one or more substituents selected from halogeno and aryl), aryl, (CH₂)₂-L¹ or the structural fragment 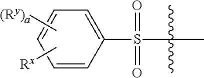
wherein R^{x} represents H or N(R^{1b})R^{2b};
R^{1b} and R^{2b} independently represent C₁₋₄ alkyl (which latter group is optionally substituted by one or more substituents selected from halogeno and aryl), aryl or (CH₂)₂-L²;
R^{y} represents halogeno or C₁₋₄ alkyl;
R^{2a} represents H, C₁₋₄ alkyl (which latter group is optionally substituted by one or more substituents selected from halogeno and aryl), aryl or (CH₂)₂-L³;
L¹, L² and L³ each represents, independently at each occurrence, a leaving group;
R³ represents halogeno or C₁₋₄ alkyl;
a represents, independently at each occurrence, an integer from 0 to 4;
X¹-X² represents C(O)—CH(Y¹), C(H)═C(Y¹), CH₂—CH(Y¹), NH—CH(Y¹), CH₂—C(O), NH—C(O) or CH(Y¹);
b represents 0 or 1;
X³-X⁴ represents CH═C(Y²), O—CH(Y²), NH—CH(Y²), O—C(O) or NH—C(O);
c represents an integer from 0 to 10;
X⁵-X⁶ represents CH₂—CH₂, CH═CH or O—CH₂; and
Y¹ and Y² independently represent, at each occurrence, H or C₁₋₄ alkyl;
or a pharmaceutically acceptable derivative thereof,
provided that at least one of the following is the case:
(a) R^{1a} represents (CH₂)₂-L¹;
(b) R^{1b} and/or R^{2b} represents (CH₂)₂-L²;
(c) R² represents (CH₂)₂-L³; and
wherein formula IX is 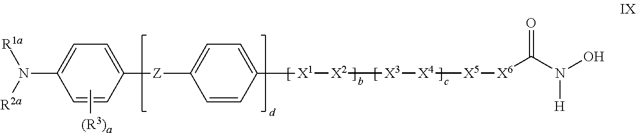
wherein
R^{1a} represents C₁₋₄ alkyl (which latter group is optionally substituted by one or more substituents selected from halogeno and aryl), aryl or (CH₂)₂-L¹;
R^{2a} represents H, C₁₋₄ alkyl (which latter group is optionally substituted by one or more substituents selected from halogeno and aryl), aryl or (CH₂)₂-L³;
L¹, L² and L³ each represents, independently at each occurrence, a leaving group;
R³ represents halogeno or C₁₋₄ alkyl;
a represents, independently at each occurrence, an integer from 0 to 4;
X¹-X² represents C(O)—CH(Y¹), C(H)═C(Y¹), CH₂—CH(Y¹), NH—CH(Y¹), CH₂—C(O), NH—C(O) or CH(Y¹);
b represents 0 or 1;
X³-X⁴ represents CH═C(Y²), O—CH(Y²), NH—CH(Y²), O—C(O) or NH—C(O);
c represents an integer from 0 to 10;
Z represents —SO₂.NH— or —NH.SO₂—;
d represents 0 or 1;
X⁵-X⁶ represents CH₂—CH₂, CH═CH or O—CH₂; and
Y¹ and Y² independently represent, at each occurrence, H or C₁₋₄ alkyl;
or a pharmaceutically acceptable derivative thereof,
provided that at least one of the following is the case:
(a) R^{1a} represents (CH₂)₂-L¹;
(c) R^{2a} represents (CH₂)₂-L³.

21-33. (canceled)

34. The pharmaceutical composition of claim 1, wherein the HDAC6 inhibitor is a compound of formula Ia, Ib, Ib′, Ic, Ic′, Id or Ie: 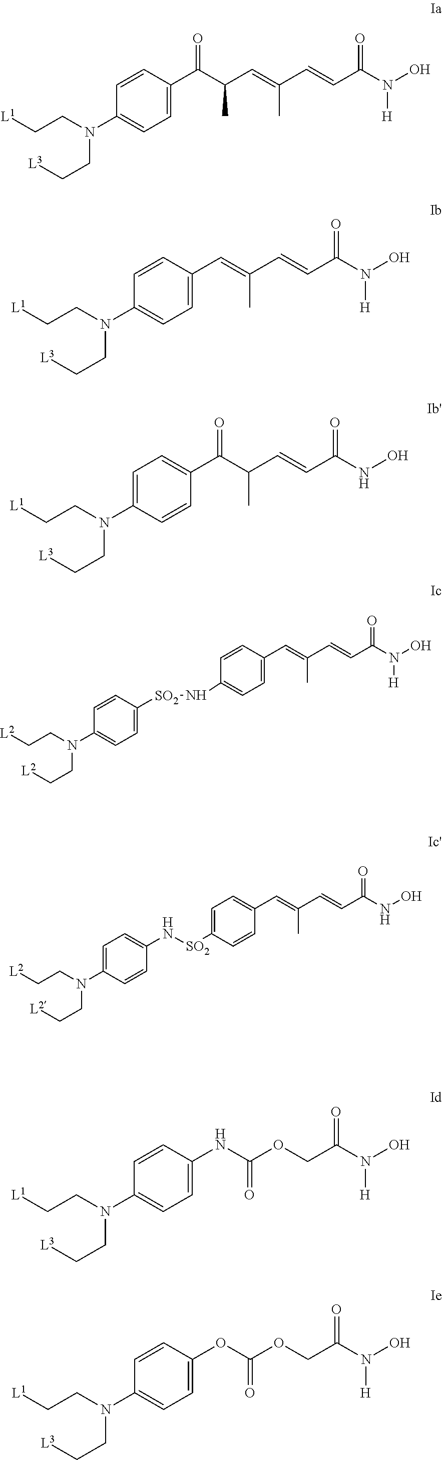
wherein L¹, L² and L³ each represents, independently at each occurrence, a leaving group, wherein L1 or L2 represents, independently at each occurrence, a halogeno group or OS(O)2R4, wherein R4 is C1-8 alkyl (optionally substituted by one or more fluoro atoms) or aryl (optionally substituted by one or more substituents selected from C1-4 alkyl, C1-4 alkoxy, NO2 and halogeno), or wherein L1 or L2 represents, independently at each occurrence, Cl, Br, I or CH3SO2O (mesyloxy), and L^{2′} represents a further leaving group, defined as for L².

35. The pharmaceutical composition of claim 1, wherein the HDAC6 inhibitor has the structure:

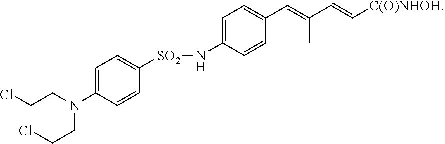

36. The pharmaceutical composition of claim 1, wherein the HDAC6 inhibitor has the structure:

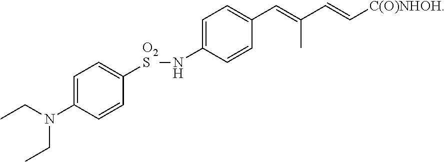

37. The pharmaceutical composition of claim 1, wherein the HDAC6 inhibitor is selected from the group comprising Tubacin, Tubastatin A, and cyclic tetrapeptide hydroxamic acids.

38. The pharmaceutical composition of claim 1, wherein the AKT inhibitor is BEZ-235, PI-103, API-2, LY294002, Wortmannin, AKT VIII, BKM120, BGT226, Everolimus, Choline kinase inhibitors, bcl-2 inhibitors, Hsp-90 inhibitors, multi-kinase inhibitors, mTOR kinase inhibitors, proteasome inhibitors, and TORC1/TORC2 inhibitors.

39. The pharmaceutical composition of claim 1, wherein the AKT inhibitor reduces AKT phosphorylation or AKT expression.

40. (canceled)

41. The pharmaceutical composition of claim 1, wherein the HDAC6 inhibitor reduces HDAC6 expression.

42. The pharmaceutical composition of claim 1, wherein the HDAC6 inhibitor is an siRNA molecule, an antisense oligonucleotide or a ribozyme.

43. The pharmaceutical composition of claim 1, wherein the AKT inhibitor specifically binds AKT protein and the HDAC6 inhibitor specifically binds HDAC6 protein.

44. The pharmaceutical composition of claim 1, wherein the AKT inhibitor specifically binds phosphorylated AKT protein.

45. (canceled)

46. The pharmaceutical composition of claim 1, wherein the HDAC6 inhibitor is an antibody, antibody fragment or derivative thereof.

47. The pharmaceutical composition of claim 1, wherein the inhibitor is a neutralising antibody, antibody fragment or derivative thereof.

48. The method of claim 8, wherein the neuro-degenerative condition is Alzheimer's disease, Parkinson's disease, Huntington's disease, Amyotrophic lateral sclerosis (ALS), spinal and bulbar muscular atrophy (SbMA), Rubinstein-Taybi syndrome, Rett syndrome or Friedreich's ataxia, and the autoimmune disease is Rheumatoid Arthritis, Myasthenia Gravis, or Multiple Sclerosis.

49. A kit comprising a HDAC6 inhibitor and an AKT inhibitor.
